(19) United States
(12) Patent Application Publication
(10) Pub. No.: US 2014/0018376 A1
(43) Pub. Date: Jan. 16, 2014

(54) Use of 1H-Quinazoline-2,4-Diones

(71) Applicants: Hans Allgeier, Loerrach (DE); Yves Auberson, Basel (CH); Thomas Blaettler, Gelterkinden (CH); David Carcache, Basel (CH); Philipp Floersheim, Basel (CH); Wolfgang Froestl, Ecublens (CH); Christel Guibourdenche, Basel (CH); Hans o Kalkman, Basel (CH); Joerg Kallen, Basel (CH); Manuel Koller, Basel (CH); Kurt Lingenhohl, Basel (CH); Henri Mattes, Basel (CH); Joachim Nozulak, Basel (CH); David Orain, Basel (CH); Johanne Renaud, Basel (CH); Christine Strohmaier, Basel (CH)

(72) Inventors: Hans Allgeier, Loerrach (DE); Yves Auberson, Basel (CH); Thomas Blaettler, Gelterkinden (CH); David Carcache, Basel (CH); Philipp Floersheim, Basel (CH); Wolfgang Froestl, Ecublens (CH); Christel Guibourdenche, Basel (CH); Hans o Kalkman, Basel (CH); Joerg Kallen, Basel (CH); Manuel Koller, Basel (CH); Kurt Lingenhohl, Basel (CH); Henri Mattes, Basel (CH); Joachim Nozulak, Basel (CH); David Orain, Basel (CH); Johanne Renaud, Basel (CH); Christine Strohmaier, Basel (CH)

(21) Appl. No.: 13/502,765

(22) PCT Filed: Oct. 20, 2010
(86) PCT No.: PCT/EP2010/065805

§ 371 Date: Apr. 19, 2012

Related U.S. Application Data
(60) Provisional application No. 61/253,336, filed on Oct. 20, 2009.

(51) Int. Cl. C07D 239/96; C07D 403/04; C07D 405/04

(52) U.S. Cl. CPC C07D 239/96; C07D 405/04; C07D 403/04
USPC 514/266.23; 514/266.3; 544/285; 514/266.24; 544/284

(57) ABSTRACT

The invention concerns the use of competitive AMPA receptor antagonists for the treatment, prevention or delay of progression of Rasmussen's encephalitis and/or certain forms of epilepsy.

### FIELD OF THE INVENTION

The present invention relates to pharmaceutical uses of 1H-quinazoline-2,4-diones, their pharmaceutically acceptable salts, and prodrugs thereof specifically for the treatment of Rasmussen's encephalitis or epilepsy, e.g. Rasmussen's encephalitis

### BACKGROUND OF THE INVENTION

Rasmussen's encephalitis is a rare, progressive neurological disorder. The typical features of Rasmussen's encephalitis are onset in childhood (usually between the ages of 14 months and 14 years) and the development of slowly progressive, neurological deterioration (including hemiparesis; cognitive impairment; dysphasia; radiological evidence of a progressive, usually unilateral, cerebral atrophy; and a pathological picture suggesting an encephalitis). Furthermore, seizures are the most common initial symptom, with approximately 20% of patients presenting with generalised or focal status epilepticus, 33% with a generalised tonic clonic seizure and the others with partial seizures. Although Rasmussen's encephalitis was described about 50 years ago, yet the cause and optimum treatment still remains unclear. Furthermore, treatment of the seizure themselves with common antiepileptic drugs has proved disappointing with most patients receiving polytherapy causing significant toxicity, but with little effect on seizures (Hart, Epileptic Disorder, 2004, 6, 133-144).

### SUMMARY OF THE INVENTION

A first aspect of the invention relates to a compound, 1H-quinazoline-2,4-diones of formula (I)

wherein

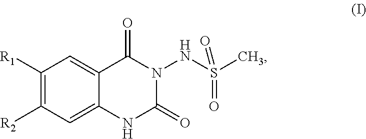

R₁ is C₁-C₆alkyl substituted by one, two or three substituents selected from hydroxy, C₁-C₆alkoxy or C₅-C₆cycloalkoxy; or

### R₁ is

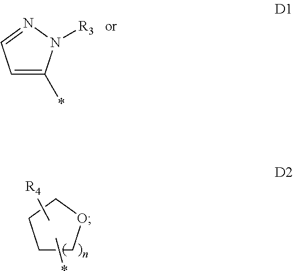

R₃ is C₁-C₆alkyl, hydroxy or C₁-C₆alkoxy-C₁-C₆alkyl;
R₄ is hydrogen or C₁-C₆alkyl;
n is 1 or 2;
R₂ is C₁-C₃alkyl or C₁-C₃-fluoroalkyl;
or a pharmaceutically acceptable salt or prodrug thereof;
may be used in the treatment, prevention or delay of progression of Rasmussen's encephalitis or epilepsy, e.g. Rasmussen's encephalitis.

A second aspect of the invention concerns a method for the treatment, prevention or delay of progression of Rasmussen's encephalitis in a subject in need of such treatment, which comprises administering to said subject a therapeutically effective amount of a 1H-quinazoline-2,4-dione of formula (I).

A third aspect of the invention concerns a method for the treatment, prevention or delay of progression of epilepsy in a subject in need of such treatment, which comprises administering to said subject a therapeutically effective amount of a 1H-quinazoline-2,4-dione of formula (I).

A fourth aspect of the invention relates to the use of a 1H-quinazoline-2,4-dione of formula (I) for the treatment (whether therapeutic or prophylactic), prevention or delay of progression of Rasmussen's encephalitis or epilepsy, e.g. Rasmussen's encephalitis.

A fifth aspect of the invention relates to a 1H-quinazoline-2,4-dione of formula (I) for the treatment, prevention or delay of progression of Rasmussen's encephalitis or epilepsy, e.g. Rasmussen's encephalitis.

A sixth aspect of the invention relates to a pharmaceutical composition comprising a 1H-quinazoline-2,4-dione of formula (I) for the treatment, prevention or delay of progression of Rasmussen's encephalitis or epilepsy, e.g. Rasmussen's encephalitis.

A seventh aspect of the invention relates to the use of a 1H-quinazoline-2,4-dione of formula (I) for the manufacture of a medicament for the treatment, prevention or delay of progression of Rasmussen's encephalitis or epilepsy, e.g. Rasmussen's encephalitis.

In a further embodiment, the invention relates to a method for the treatment, prevention or delay of progression of Rasmussen's encephalitis in a subject in need of such treatment, which comprises administering to said subject a therapeutically effective amount of a compound of formula (I), wherein said subject is selected among those having an abnormal serum level of anti-GluR3 autoantibodies.

In a further embodiment, the invention relates to a method for the treatment, prevention or delay of progression of Rasmussen's encephalitis in a subject in need of such treatment, which comprises administering to said subject a therapeutically effective amount of a compound of formula (I), wherein said subject is selected among those having an abnormal serum level of anti-GluR3 autoantibodies with agonistic activity.

A further aspect of the invention is a method for the treatment, prevention or delay of progression of epilepsy in a subject in need of such treatment, which comprises administering to said subject a therapeutically effective amount of a compound of formula (I), wherein said subject is selected among those having an abnormal serum level of anti-GluR3 autoantibodies.

A further aspect of the invention is a method for the treatment, prevention or delay of progression of epilepsy in a subject in need of such treatment, which comprises administering to said subject a therapeutically effective amount of a compound of formula (I), wherein said subject is selected among those having an abnormal serum level of anti-GluR3 autoantibodies with agonistic activity.

A further aspect of the invention is a method of screening a subject to determine whether they are likely to respond to treatment with a 1H-quinazoline-2,4-dione of formula (I) comprising the steps of:

(i) obtaining a biological sample from said subject,
(ii) detecting the presence of anti-GluR3 autoantibodies, and
(iii) comparing the titre of anti-GluR3 autoantibodies with a control value,
wherein an increased titre of anti-GluR3 autoantibodies compared to the control value indicates an increased likelihood of response to treatment.

A further aspect of the invention is a method for monitoring the treatment of epilepsy or Rasmussen's encephalitis, e.g. Rasmussen's encephalitis, in a subject comprising the steps of:

(i) obtaining a first biological sample from said subject,
(ii) detecting a first titre of anti-GluR3 autoantibodies,
(iii) treating the subject with a 1H-quinazoline-2,4-dione of formula (I),
(iv) obtaining a biological tissue sample from said subject,
(v) detecting a second titre of anti-GluR3 autoantibodies, and
(vi) comparing the first and second titres of anti-GluR3 autoantibodies, wherein a change in the second antibody titre compared to the first antibody titre indicates that the treatment is effective.

Of course, in certain aspects, the biological sample may have already been obtained from a subject, thus the invention further provides: a method of screening a subject to determine whether they are likely to respond to treatment with a 1H-quinazoline-2,4-dione of formula (I) comprising the steps of:

(i) detecting the presence of anti-GluR3 autoantibodies in a biological sample obtained from a subject, and
(ii) comparing the titre of anti-GluR3 autoantibodies with a control value,
wherein an increased titre of anti-GluR3 autoantibodies compared to the control value indicates an increased likelihood of response to treatment.

The invention also provides:

a method for monitoring the treatment of epilepsy or Rasmussen's encephalitis, e.g. Rasmussen's encephalitis, in a subject comprising the steps of:
(i) detecting a first titre of anti-GluR3 autoantibodies from a first biological sample obtained from the subject,
(ii) treating the subject with a 1H-quinazoline-2,4-dione of formula (I),
(iii) detecting a second titre of anti-GluR3 autoantibodies from a second biological sample obtained from the subject, and
(iv) comparing the first and second titres of anti-GluR3 autoantibodies, wherein a change in the second antibody titre compared to the first antibody titre indicates that the treatment is effective.

### DETAILED DESCRIPTION OF THE INVENTION

The invention relates to a compound, 1H-quinazoline-2,4-diones of formula (I)

wherein

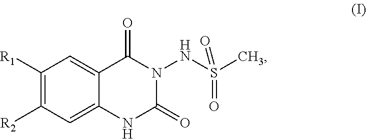

R₁ is C₁-C₆alkyl substituted by one, two or three substituents selected from hydroxy, C₁-C₆alkoxy or C₅-C₆cycloalkoxy; C₅-C₆cycloalkyl substituted by one, two or three substituents selected from hydroxy, C₁-C₆alkoxy or C₅-C₆cycloalkoxy; or

### R₁ is

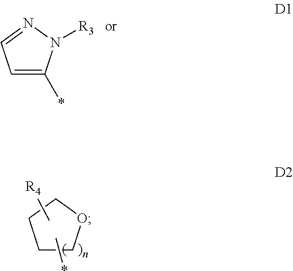

R₃ is C₁-C₆alkyl, hydroxy or C₁-C₆alkoxy-C₁-C₆alkyl;
R₄ is hydrogen or C₁-C₆alkyl;
n is 1 or 2;
R₂ is C₁-C₃alkyl or C₁-C₃-fluoroalkyl;
their pharmaceutically acceptable salts, and their prodrugs thereof;
for use in a method for the treatment, prevention or delay of progression of Rasmussen's encephalitis or epilepsy, e.g. Rasmussen's encephalitis.

The compound of formula (I) is a competitive AMPA antagonist. It is well understood that allosteric (non-competitive) antagonists provide an insurmountable blockade of AMPA receptors, potentially preventing any AMPA receptor-mediated neurotransmission at the synapse. In contrast, a high concentration of glutamate at the synapse can still activate the post-synaptic membrane in the presence of a competitive AMPA antagonist (albeit with a lower efficacy). Competitive AMPA antagonists may therefore exhibit an improved safety profile, as they will not fully block neurotransmission, but instead reduce the exaggerated glutamate signaling observed in some neurological disease, e.g. epilepsy.

Compounds of the formula (I) not only block AMPA-induced glutamate release from activated astrocytes but after oral dosing also suppress epilepsy seizures in epilepsy or in Rasmussen encephalitis.

The compound of the invention of formula (I) in addition to the advantage of being a competitive AMPA antagonist receptor inhibitor, presents also the advantage of being a selective competitive AMPA antagonist. Furthermore the compound of the invention of formula (I) is capable of penetrating the blood brain barrier and may be formulated in an oral dosage form.

In the present specification, the following definitions shall apply if no specific other definition is given:

Bonds with the asterisk (*) denote point of binding to the rest of the molecule.

“C₁-C₆alkyl” represents a straight-chain or branched-chain alkyl group; for example, methyl, ethyl, n- or iso-propyl, n-, iso-, sec- or tert-butyl, n-pentyl, n-hexyl, with particular preference given to methyl, ethyl, n-propyl and iso-propyl.

“C₅-C₆cycloalkyl” represents cyclopentyl or cyclohexyl; preferably cyclopentyl.

Each alkyl/cycloalkyl-part of “alkoxy”, “cycloalkoxy”, “alkoxyalkyl” and “fluoroalkyl” shall have the same meaning as described in the above-mentioned definitions of “alkyl”/“cycloalkyl”.

“C₁-C₃-fluoroalkyl” preferably represents trifluoromethyl, difluoromethyl or fluoromethyl.

It will be understood that any discussion of methods or references to the active ingredients includes said active ingredient in free form and in form of a pharmaceutically acceptable salt. If the active ingredients have, for example, at least one acidic center (for example COOH) they can form salts with bases. The active ingredient or a pharmaceutically acceptable salt thereof may also be used in the form of a hydrate or may include other solvents used for crystallization.

A “pharmaceutically acceptable salt” is intended to mean a salt of a free base/free acid of a compound represented by formula (I) that is not toxic, biologically intolerable, or otherwise biologically undesirable. Preferred pharmaceutically acceptable salts are those that are pharmacologically effective and suitable for contact with the tissues of patients without undue toxicity, irritation, or allergic response. Such salts are known in the field (e.g. S. M. Berge, et al, “Pharmaceutical Salts”, J. Pharm. Sd., 1977, 66:1-19; and “Handbook of Pharmaceutical Salts, Properties, Selection, and Use”, Stahl, R H., Wermuth, C. G., Eds.; Wiley-VCH and VHCA: Zurich, 2002).

In one embodiment of the invention, the 1H-quinazoline-2,4-diones of formula (I) is used in free form.

The 1H-quinazoline-2,4-diones of formula (I), their manufacture and their use as competitive AMPA receptor antagonists are known from WO 2006/108591 or can be prepared analogously to said reference. WO 2006/108591 is incorporated herein by reference.

On account of asymmetrical carbon atom(s) that may be present in the 1H-quinazoline-2,4-diones of formula (I) and their pharmaceutically acceptable salts, the compounds may exist in optically active form or in form of mixtures of optical isomers, e.g. in form of racemic mixtures or diastereomeric mixtures. All optical isomers and their mixtures, including racemic mixtures, are part of the present invention.

In one embodiment of the invention, the 1H-quinazoline-2,4-dione of formula (I) is a compound, wherein R₁ is C₁-C₆alkyl substituted by one, two or three substituents selected from hydroxy, C₁-C₆alkoxy or C₅-C₆cycloalkoxy; and R₂ is C₁-C₃alkyl or C₁-C₃-fluoroalkyl.

In one embodiment of the invention, the 1H-quinazoline-2,4-dione of formula (I) is a compound, wherein R₁ is

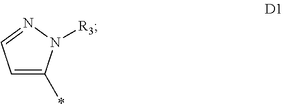

R₃ is C₁-C₆alkyl, hydroxy or C₁-C₆alkoxy-C₁-C₆alkyl; and R₂ is C₁-C₃alkyl or C₁-C₃-fluoroalkyl.

In one embodiment of the invention, the 1H-quinazoline-2,4-dione of formula (I) is a compound, wherein R₁ is

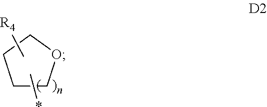

R₄ is hydrogen or C₁-C₆alkyl; n is 1 or 2; and R₂ is C₁-C₃alkyl or C₁-C₃-fluoroalkyl.

In one embodiment of the invention, the 1H-quinazoline-2,4-dione of formula (I) is a compound selected from the group consisting of
A-1: N-[6-(1-Hydroxy-ethyl)-2,4-dioxo-7-trifluoromethyl-1,4-dihydro-2H-quinazolin-3-yl]-methanesulfonamide;
A-2: N-[6-(1-Methoxy-ethyl)-2,4-dioxo-7-trifluoromethyl-1,4-dihydro-2H-quinazolin-3-yl]-methanesulfonamide;
A-3: N-[6-(1-Hydroxy-propyl)-2,4-dioxo-7-trifluoromethyl-1,4-dihydro-2H-quinazolin-3-yl]-methanesulfonamide;
A-4: N-[6-(1-Isopropoxy-ethyl)-2,4-dioxo-7-trifluoromethyl-1,4-dihydro-2H-quinazolin-3-yl]-methanesulfonamide;
A-5: N-[6-(1-Ethoxy-ethyl)-2,4-dioxo-7-trifluoromethyl-1,4-dihydro-2H-quinazolin-3-yl]-methanesulfonamide;
A-6: N-[2,4-Dioxo-6-(1-propoxy-propyl)-7-trifluoromethyl-1,4-dihydro-2H-quinazolin-3-yl]-methanesulfonamide;
A-7: N-[6-(1-isopropoxy-propyl)-2,4-dioxo-7-trifluoromethyl-1,4-dihydro-2H-quinazolin-3-yl]-methanesulfonamide;
A-8: N-[7-Difluoromethyl-6-(1-ethoxy-ethyl)-2,4-dioxo-1,4-dihydro-2H-quinazolin-3-yl]-methanesulfonamide;
A-9: N-[2,4-Dioxo-6-(1-propoxy-ethyl)-7-trifluoromethyl-1,4-dihydro-2H-quinazolin-3-yl]-methanesulfonamide;
A-10: N-[6-(1-Butoxy-ethyl)-2,4-dioxo-7-trifluoromethyl-1,4-dihydro-2H-quinazolin-3-yl]-methanesulfonamide;
A-11: N-[6-(1-Isobutoxy-ethyl)-2,4-dioxo-7-trifluoromethyl-1,4-dihydro-2H-quinazolin-3-yl]-methanesulfonamide;
A-12: N-[6-(1-methoxy-butyl)-2,4-dioxo-7-trifluoromethyl-1,4-dihydro-2H-quinazolin-3-yl]-methanesulfonamide;
A-13: N-[6-(1-Ethoxy-propyl)-2,4-dioxo-7-trifluoromethyl-1,4-dihydro-2H-quinazolin-3-yl]-methanesulfonamide;
A-14: N-[6-(1-Cyclopentyloxy-ethyl)-2,4-dioxo-7-trifluoromethyl-1,4-dihydro-2H-quinazolin-3-yl]-methanesulfonamide;
A-15: N-[6-(1-Hydroxy-butyl)-2,4-dioxo-7-trifluoromethyl-1,4-dihydro-2H-quinazolin-3-yl]-methanesulfonamide;
A-16: N-[6-(1-Methoxy-2-methyl-propyl)-2,4-dioxo-7-trifluoromethyl-1,4-dihydro-2H-quinazolin-3-yl]-methanesulfonamide;
A-17: N-[6-(3-Hydroxy-propyl)-2,4-dioxo-7-trifluoromethyl-1,4-dihydro-2H-quinazolin-3-yl]-methanesulfonamide;
A-18: N-[6-(1-Hydroxy-3-methoxy-propyl)-2,4-dioxo-7-trifluoromethyl-1,4-dihydro-2H-quinazolin-3-yl]-methanesulfonamide;
A-19: N-[6-(1-Hydroxy-2-methyl-propyl)-2,4-dioxo-7-trifluoromethyl-1,4-dihydro-2H-quinazolin-3-yl]-methanesulfonamide;
B-1: N-[2,4-Dioxo-6-(tetrahydro-pyran-2-yl)-7-trifluoromethyl-1,4-dihydro-2H-quinazolin-3-yl]-methanesulfonamide;
B-2: N-[2,4-Dioxo-6-(tetrahydro-furan-2-yl)-7-trifluoromethyl-1,4-dihydro-2H-quinazolin-3-yl]-methanesulfonamide;
B-3: N-[2,4-Dioxo-6-(tetrahydro-furan-3-yl)-7-trifluoromethyl-1,4-dihydro-2H-quinazolin-3-yl]-methanesulfonamide;
C-1: N-{7-Isopropyl-6-[2-(2-methoxy-ethyl)-2H-pyrazol-3-yl]-2,4-dioxo-1,4-dihydro-2H-quinazolin-3-yl}-methanesulfonamide;
C-2: N-[6-(2-Isopropyl-2H-pyrazol-3-yl)-2,4-dioxo-7-trifluoromethyl-1,4-dihydro-2H-quinazolin-3-yl]-methanesulfonamide;
C-3: N-[7-Fluoromethyl-6-(2-isopropyl-2H-pyrazol-3-yl)-2,4-dioxo-1,4-dihydro-2H-quinazolin-3-yl]-methanesulfonamide;
C-4: N-{6-[2-(2-Methoxy-ethyl)-2H-pyrazol-3-yl]-2,4-dioxo-7-trifluoromethyl-1,4-dihydro-2H-quinazolin-3-yl}-methanesulfonamide;
C-5: N-[6-(2-Hydroxy-2H-pyrazol-3-yl)-2,4-dioxo-7-trifluoromethyl-1,4-dihydro-2H-quinazolin-3-yl]-methanesulfonamide;
C-6: N-[7-Ethyl-6-(2-isopropyl-2H-pyrazol-3-yl)-2,4-dioxo-1,4-dihydro-2H-quinazolin-3-yl]-methanesulfonamide;
C-7: N-[7-Isopropyl-6-(2-methyl-2H-pyrazol-3-yl)-2,4-dioxo-1,4-dihydro-2H-quinazolin-3-yl]-methanesulfonamide;
C-8: N-[7-Isopropyl-6-(2-isopropyl-2H-pyrazol-3-yl)-2,4-dioxo-1,4-dihydro-2H-quinazolin-3-yl]-methanesulfonamide;
C-9: N-[7-Difluoromethyl-6-(2-methyl-2H-pyrazol-3-yl)-2,4-dioxo-1,4-dihydro-2H-quinazolin-3-yl]-methanesulfonamide;
C-10: N-[7-Difluoromethyl-6-(2-isopropyl-2H-pyrazol-3-yl)-2,4-dioxo-1,4-dihydro-2H-quinazolin-3-yl]-methanesulfonamide;
C-11: N-[7-Ethyl-6-(2-methyl-2H-pyrazol-3-yl)-2,4-dioxo-1,4-dihydro-2H-quinazolin-3-yl]-methanesulfonamide;
C-12: N-[7-Ethyl-6-(2-ethyl-2H-pyrazol-3-yl)-2,4-dioxo-1,4-dihydro-2H-quinazolin-3-yl]-methanesulfonamide;
C-13: N-[7-Fluoromethyl-6-(2-methyl-2H-pyrazol-3-yl)-2,4-dioxo-1,4-dihydro-2H-quinazolin-3-yl]-methanesulfonamide;
C-14: N-[7-(1-fluoro-ethyl)-6-(2-methyl-2H-pyrazol-3-yl)-2,4-dioxo-1,4-dihydro-2H-quinazolin-3-yl]-methanesulfonamide;
C-15: N-[7-(1,1-difluoro-ethyl)-6-(2-methyl-2H-pyrazol-3-yl)-2,4-dioxo-1,4-dihydro-2H-quinazolin-3-yl]-methanesulfonamide;
C-16: N-[7-(1,1-difluoro-ethyl)-6-(2-isopropyl-2H-pyrazol-3-yl)-2,4-dioxo-1,4-dihydro-2H-quinazolin-3-yl]methanesulfonamide;
C-17: N-[7-(1-fluoro-ethyl)-6-(2-isopropyl-2H-pyrazol-3-yl)-2,4-dioxo-1,4-dihydro-2H-quinazolin-3-yl]-methanesulfonamide; and
C-18: N-[6-(2-Methyl-2H-pyrazol-3-yl)-2,4-dioxo-7-trifluoromethyl-1,4-dihydro-2H-quinazolin-3-yl]-methanesulfonamide.

The compounds of the invention, including the specific exemplified compounds, may be prepared by any suitable method, e.g. as described in WO 2006/108591.

In one embodiment of the invention, the 1H-quinazoline-2,4-dione of formula (I) is a compound selected from the group consisting of compound A-1, A-2, A-3, A-4, A-5, A-6, A-7, A-8, A-9, A-10, A-11, A-12, A-13, A-14, A-15, A16, A17, A-18 and A-19.

In one embodiment of the invention, the 1H-quinazoline-2,4-dione of formula (I) is a compound selected from the group consisting of compound B-1, B-2 and B-3.

In one embodiment of the invention, the 1H-quinazoline-2,4-dione of formula (I) is a compound selected from the group consisting of compound C-1, C-2, C-3, C-4, C-5, C-6, C-7, C-8, C-9, C-10, C-11, C-12, C-13, C-14, C-15, C-16, C-17 and C-18.

Advantageous compounds of the invention, i.e., the 1H-quinazoline-2,4-diones of formula (I), should be well absorbed from the gastrointestinal tract, penetrate the blood brain barrier, be sufficiently metabolically stable and possess favorable pharmacokinetic properties.

Preferred compounds, having superior bioavailibility are 1H-quinazoline-2,4-dione of formula (I) selected from the group consisting of compounds: A-1, A-2, A-3, A-4, A-5, A-6, A-7, A-13, A-14, A-15, A-18, B-2, B-3, C-1, C-2, C-3, C-4, C-5, C-6, C-7, C-8, C-9, C-10, C-11, C-12, C-15, C-16, C-17 and C-18.

More preferred compounds, having superior bioavailibility are 1H-quinazoline-2,4-dione of formula (I) selected from the group consisting of compounds: A-1, A-2, A-3, A-4, A-5, A-7, A-15, B-2, B-3, C-1, C-2, C-3, C-6, C-7, C-8, C-9, C-10, C-11, C-12, C-15, C-17 and C-18.

Further more preferred compounds, having superior bioavailibility are 1H-quinazoline-2,4-dione of formula (I) selected from the group consisting of compounds: A-2, A-3, A-4, A-5 B-2, C-2, C-3, C-7, C-9, C-10, C-11, C-15 and C-18.

Most preferred compounds, having superior bioavailibility are 1H-quinazoline-2,4-dione of formula (I) selected from the group consisting of compounds: A-2, A-5, B-2, C-7, C-9 and C-11.

Compounds for use in the present invention are either obtained in the free form, as a salt thereof, or as prodrug derivatives thereof.

The term “prodrug” as used herein relates to a compound, which converts in vivo into a compound used in the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. The term “prodrug,” as used herein, represents in particular compounds which are transformed in vivo to the parent compound, for example, by hydrolysis in blood, for example as described in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), and “The Organic Chemistry of Drug Design and Drug Action”, 2^{nd} Edition, R B Silverman (particularly Chapter 8, pages 497 to 557), Elsevier Academic Press, 2004.

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. alkyl esters |
| Alcohol | Esters, including e.g. sulfates and phosphates |
| | as well as carboxylic acid esters |
| Amine | Amides, carbamates, imines, enamines, |
| Carbonyl (aldehyde, | Imines, oximes, acetals/ketals, enol esters, |
| ketone) | oxazolidines and thiazoxolidines |

Prodrugs also include compounds convertible to the active drug by an oxidative or reductive reaction. As examples may be mentioned

### Oxidative Activation

### N- and O-dealkylation

Oxidative deamination

### N-oxidation

### Epoxidation

### Reductive Activation

Azo reduction
Sulfoxide reduction
Disulfide reduction
Bioreductive alkylation
Nitro reduction.

Each of the above described reactions and/or reaction steps can be used individually or in combination in a method to prepare a AMPA-inhibitor or a prodrug thereof.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like.

The term “hydrate” refers to the complex where the solvent molecule is water. The compounds of the present invention, including salts, hydrates and solvates thereof, may inherently or by design form polymorphs.

Preferred prodrugs of the invention should be well absorbed from the gastrointestinal tract, be transformed into the parent compound (or active principle, being the compound that in-vivo acts as AMPA receptor antagonist), the parent compound should be sufficiently metabolically stable and possess favorable pharmacokinetic properties.

Further preferred prodrugs of the invention lead to an oral bioavailability of the parent compound which is comparable to the bioavailability when administered as a drug.

Further preferred prodrugs of the invention exhibit increased oral bioavailability compared to the parent compound when administered as a drug. Oral bioavailability may manifest itself in different ways: (i) a biological effect may be achieved after oral administration when the parent compound is less effective upon oral administration, (ii) an earlier onset of action upon oral administration, (iii) a lower dose needed to achieve the same effect, (iv) a higher effect achieved by the same dose or (v) a prolonged action at the same dose.

Further preferred prodrugs of the invention are transformed into parent compounds which in-vivo bind potently to AMPA receptors whilst showing little affinity for other receptors.

Some prodrugs of the invention are transformed into parent compounds which also show antagonistic activity at kainate receptors. Besides such dual activity, showing little affinity for other receptors is a preferred feature.

Further prodrugs of the invention—when the active principle is targeted against receptors in the central nervous system—are transformed into parent compounds that cross the blood brain barrier freely.

Further prodrugs of the invention—when the active principle is targeted selectively against receptors in the peripheral nervous system—are transformed into parent compounds that do not cross the blood brain barrier.

Prodrugs, parent compounds and released pro-moieties should be non-toxic and demonstrate few side-effects.

Furthermore, the ideal prodrug of the invention will be able to exist in a physical form that is stable, non-hygroscopic and easily formulated.

The higher oral bioavailability of the compounds for use in the invention may give rise to the following beneficial effects relating to less bioavailable compounds: (i) an enhanced biological effect may be achieved after oral administration; (ii) an earlier onset of action may be observed following oral administration; (iii) a lower dose may be needed to achieve the same effect; (iv) a higher effect may be achieved by the same dose or (v) a prolonged action may be observed at the same dose.

Preferably the compound for use in the invention when tested in-vivo potently binds to AMPA receptors whilst showing little affinity for other receptors.

The term “subject” as used herein refers to a human being, especially to a patient being diagnosed with Rasmussen's encephalitis or epilepsy, or who is suspected of having Rasmussen's encephalitis or epilepsy.

The term “treatment” as used herein refers to any type of treatment that imparts a benefit to a subject affected with a disease, e.g. a patient diagnosed with a disease, including improvement in the condition of the subject (e.g. in one or more symptoms) or prevention/delay of the onset/progression of the disease (e.g. prophylactic treatment). In the case of a patient being diagnosed with Rasmussen's encephalitis, treatment typically comprise a reduction in the symptoms associated with Rasmussen's encephalitis, including for example, although not limited to, a reduction in number and severity of seizures, a reduction of cognitive impairment, a reduction of dysphasia, a reduction of brain areas showing atrophy or a pathological picture suggesting an encephalitis. In the case of a patient being diagnosed with epilepsy, treatment typically comprise a reduction in the symptoms associated with epilepsy, including a reduction in number and severity of seizures.

The term “therapeutically effective amount” as used herein typically refers to a drug amount which, when administered to a subject, is sufficient to provide a therapeutic benefit, e.g. is sufficient for treating, preventing or delaying the progression of epilepsy or Rasmussen's encephalitis (e.g. the amount provides an amelioration of symptoms, e.g. it leads to a reduction in number and severity of seizures) or is sufficient for treating, preventing or delaying the progression of epilepsy.

For the above-mentioned indications (the conditions and disorders) the appropriate dosage will vary depending upon, for example, the compound employed, the host, the mode of administration and the nature and severity of the condition being treated. However, in general, satisfactory results in animals are indicated to be obtained at a daily dosage of from about 0.01 to about 100 mg/kg body weight, preferably from about 1 to about 30 mg/kg body weight, e.g. 10 mg/kg. In larger mammals, for example humans, an indicated daily dosage is in the range from about 0.1 to about 1000 mg, preferably from about 1 to about 400 mg, most preferably from about 10 to about 100 mg of a 1H-quinazoline-2,4-dione of formula (I) conveniently administered, for example, in divided doses up to four times a day. In one embodiment, about 100 mg of a 1H-quinazoline-2,4-dione of formula (I) is administered daily. In a further embodiment, about 200 mg of a 1H-quinazoline-2,4-dione of formula (I) is administered daily.

For use according to the invention, the 1H-quinazoline-2,4-diones of formula (I) may be administered as single active agent or in combination with one or more other active agents, in any usual manner, e.g. orally, for example in the form of tablets, capsules or drinking solutions; rectally, for example in the form of suppositories; intravenously, for example in the form of injection solutions or suspensions; or transdermally, for example in the form of a patch.

In one embodiment, the manner of administration is oral administration, for example in the form of a tablet, capsule or drinking solution.

In one embodiment, the manner of administration is rectal administration, for example in the form of a suppository.

In one embodiment, the manner of administration is transdermal administration, for example in the form of a patch.

In one preferred embodiment, the manner of administration is oral administration.

Preferred pharmaceutical compositions comprise a 1H-quinazoline-2,4-diones of formula (I) in association with at least one pharmaceutical carrier or diluent. Such compositions may be manufactured in conventional manner. Unit dosage forms may contain, for example, from about 2.5 to about 250 mg, preferably from about 2.5 to about 200 mg, more preferably from about 2.5 to about 100 mg, still more preferably from about 2.5 to about 50 mg and still more preferably from about 2.5 to about 25 mg, of one or more of the 1H-quinazoline-2,4-diones of formula (I).

The pharmaceutical compositions according to the invention are compositions for enteral administration, such as oral or rectal administration; or parenteral administration, such as intramuscular, intravenous, and nasal or transdermal administration, to warm-blooded animals (human beings and animals) that comprise an effective dose of the pharmacological active ingredient alone or together with a significant amount of a pharmaceutically acceptable carrier. The dose of the active ingredient depends on the species of warm-blooded animal, body weight, age and individual condition, individual pharmacokinetic data, the disease to be treated and the mode of administration.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragees, tablets or capsules.

The pharmaceutical compositions of the present invention are prepared in a manner known per se, for example by means of conventional dissolving, lyophilizing, mixing, granulating or confectioning processes. Such processes are exemplified in WO 2005/079802, WO 2003/047581, WO 2004/000316, WO 2005/044265, WO 2005/044266, WO 2005/044267, WO 2006/114262 and WO 2007/071358.

Compositions for transdermal administration are described in Remington's Pharmaceutical Sciences 16^{th} Edition Mack; Sucker, Fuchs and Spieser, Pharmazeutische Technologie, 1^{st} Edition, Springer.

In one embodiment, the invention relates to a method for the treatment, prevention or delay of progression of epilepsy or Rasmussen's encephalitis in a subject in need of such treatment, which comprises administering to said subject a therapeutically effective amount of a compound of formula (I), wherein said subject is selected among those having an abnormal serum level of anti-GluR3 autoantibodies.

In one embodiment, the invention relates to a method for the treatment, prevention or delay of progression of epilepsy or Rasmussen's encephalitis in a subject in need of such treatment, which comprises administering to said subject a therapeutically effective amount of a compound of formula (I), wherein said subject is selected among those having an abnormal serum level of anti-GluR3 autoantibodies with agonistic activity.

A further aspect of the invention is a method for the treatment, prevention or delay of progression of epilepsy in a subject in need of such treatment, which comprises administering to said subject a therapeutically effective amount of a compound of formula (I), wherein said subject is selected among those having an abnormal serum level of anti-GluR3 autoantibodies.

A further aspect of the invention is a method for the treatment, prevention or delay of progression of epilepsy in a subject in need of such treatment, which comprises administering to said subject a therapeutically effective amount of a compound of formula (I), wherein said subject is selected among those having an abnormal serum level of anti-GluR3 autoantibodies with agonistic activity.

A further aspect of the invention is a method of screening a subject to determine whether they are likely to respond to treatment with a 1H-quinazoline-2,4-dione of formula (I) comprising the steps of:

(i) obtaining a biological sample from said subject,
(ii) detecting the presence of anti-GluR3 autoantibodies, and
(iii) comparing the titre of anti-GluR3 autoantibodies with a control value,
wherein an increased titre of anti-GluR3 autoantibodies compared to the control value indicates an increased likelihood of response to treatment.

A further aspect of the invention is a method for monitoring the treatment of epilepsy or Rasmussen's encephalitis in a subject comprising the steps of:

(i) obtaining a first biological sample from said subject,
(ii) detecting a first titre of anti-GluR3 autoantibodies,
(iii) treating the subject with a 1H-quinazoline-2,4-dione of formula (I),
(iv) obtaining a biological tissue sample from said subject,
(v) detecting a second titre of anti-GluR3 autoantibodies, and
(vi) comparing the first and second titres of anti-GluR3 autoantibodies, wherein a change in the second antibody titre compared to the first antibody titre indicates that the treatment is effective.

Of course, in certain aspects, the biological sample may have already been obtained from a subject, thus the invention provides:

a method of screening a subject to determine whether they are likely to respond to treatment with a 1H-quinazoline-2,4-dione of formula (I) comprising the steps of:

(i) detecting the presence of anti-GluR3 autoantibodies in a biological sample obtained from a subject, and
(ii) comparing the titre of anti-GluR3 autoantibodies with a control value,
wherein an increased titre of anti-GluR3 autoantibodies compared to the control value indicates an increased likelihood of response to treatment.

The invention also provides:

a method for monitoring the treatment of epilepsy or Rasmussen's encephalitis in a subject comprising the steps of:
(i) detecting a first titre of anti-GluR3 autoantibodies from a first biological sample obtained from the subject,
(ii) treating the subject with a 1H-quinazoline-2,4-dione of formula (I),
(iii) detecting a second titre of anti-GluR3 autoantibodies from a second biological sample obtained from the subject, and
(iv) comparing the first and second titres of anti-GluR3 autoantibodies, wherein a change in the second antibody titre compared to the first antibody titre indicates that the treatment is effective.

The monitoring of the subject using such a method will also allow the physician to tailor the treatment of the subject. Thus, if the autoantibody titre increases, the dosage of a 1H-quinazoline-2,4-dione of formula (I) may be increased, or vice versa. Conversely, if the autoantibody titre decreases, the dosage of a 1H-quinazoline-2,4-dione of formula (I) may be decreased, or vice versa.

In one embodiment, the anti-GluR3 autoantibodies are anti-GluR3B autoantibodies.

As used herein, the term “autoantibodies” refers to antibodies formed in response to an immune reaction and reacting against an antigenic constituent of the subject's own proteins, cells or tissues. The autoantibody may be of the isotype IgA, IgD, IgE, IgG, or IgM. In one embodiment, the autoantibody isotype is IgG or IgM. Different assays may be required to optimally detect the different isotypes.

As used herein, the term “anti-GluR3 autoantibodies” refers to autoantibodies that bind to GluR3 antigen. Such binding must be measurably and significantly different from a non-specific interaction.

The GluR3 gene belongs to the gene family comprising GluR1, GluR2, GluR3 and GluR4, which are all coding for subunits of homomeric or heteromeric AMPA receptors. Said AMPA receptors are ionotrophic glutamate receptors found in the central nervous system. GluR3B is a peptide of 24 amino acids. Both human (NEYERFVPFSDQQISNDSASSENR—SEQ ID NO: 6) and rodent (NEYERFVPFSDQQISNDSSSSENR—SEQ ID NO: 7) forms of GluR3B are known to exist.

The serum level of antibodies, such as anti-GluR3 autoantibodies or anti-GluR3 autoantibodies with agonistic activity, can be determined from a biological sample, for example a blood sample or a serum sample, of a subject in need of treatment. In a preferred embodiment, a serum sample is obtained from a subject in need of treatment. Said biological sample may be pre-treated prior to determining the level of anti-GluR3 auto-antibodies. Pre-treatment may consist of purification steps for enriching the sample with anti-GluR3 antibodies or depletion steps, for example, for removing non-immunoglobulin materials. In a further embodiment, the sample may be purified to remove any non-IgG materials. In a further embodiment, the sample may be purified to remove any non-IgM materials.

The presence or the level of anti-GluR3 autoantibodies in a biological sample, e.g. serum sample, can be determined using standard methods, such as Western Blot, immunoprecipitation, surface plasmon resonance analysis (e.g. using BiaCore) or ELISA. Examples of specific methods using ELISA for detecting human anti-GluR3 autoantibodies in serum samples are described in the Examples. In one embodiment, the assay is an ELISA assay. In one embodiment, the ELISA assay uses a chaotropic buffer.

For the detection of autoantibodies, a peptide to which the antibodies bind may be used. For example, if an ELISA assay is carried out, the peptide (or substrate) may be immobilised to the surface of a multi-well plate. Streptavidin coated plates combined with biotin tagged peptide may be used for this purpose. The serum sample may then be added to the multi-well plate, allowing autoantibodies to selectively bind to the peptide. The peptides of SEQ ID NOs: 1-4, 6 and 7, are suitable peptides for such uses. In one embodiment, the peptide of SEQ ID NO: 6 is used in the assay.

As used herein, the serum level of anti-GluR3 autoantibodies is considered abnormal when the amount of anti-GluR3 antibodies detected in a test sample from a subject is significantly higher than the amount (control value) detected in the control samples from a control population, for example, serum samples from healthy donors.

In one embodiment, the serum level of anti-GluR3 autoantibodies is considered abnormal when the level of anti-GluR3 autoantibodies detected in the serum sample of a subject is at least equal to the corresponding mean level+2 fold or greater (e.g. 3, 4, 5 fold or greater) the standard deviations measured in the control samples, for example, from serum samples of healthy donors or patients not suffering from Rasmussen's encephalitis or epilepsy or related disorders.

In another embodiment, the serum level of anti-GluR3 autoantibodies is considered abnormal when the level of anti-GluR3 autoantibodies detected in the serum sample of a subject is 50% or more (i.e. 60, 70, 80, 90, 100, 200, 500% or more) than that of the average measured in control samples from healthy donors or subjects not suffering from Rasmussen's encephalitis or epilepsy or related disorders.

In another embodiment, the level of anti-GluR3 antibodies is considered abnormal when the amount of anti-GluR3 antibodies is a detectable amount in the test sample whereas no significant amount of anti-GluR3 antibodies is detected in the control samples (e.g. from healthy donors or patients not suffering from Rasmussen's encephalitis or epilepsy or related disorders).

In one specific embodiment, a subject with abnormal level of anti-GluR3 autoantibodies is a subject suffering from Rasmussen's encephalitis and having a serum level of anti-GluR3 autoantibodies superior to 1 μg/ml, 10 μg/ml, 100 μg/ml or 500 μg/ml.

In another specific embodiment, a subject with abnormal level of anti-GluR3 autoantibodies is a subject suffering from epilepsy and having a serum level of anti-GluR3 autoantibodies superior to 1 μg/ml, 10 μg/ml, 100 μg/ml or 500 μg/ml.

The presence or the level of anti-GluR3 autoantibodies with agonistic activity in a biological sample, e.g. serum sample, can be determined, for example, as described below. One method for detecting anti-GluR3 autoantibodies with agonistic activity from serum sample of a subject is described in the Examples. Any other methods for detecting functional activity of GluR3 receptor induced in the presence of the test antibodies can be used.

As used herein, the term “GluR3 receptor” refers to an ionotrophic glutamate receptor comprising at least one GluR3 subunit.

Preferably, anti-GluR3 autoantibodies with agonistic activity from serum are first purified (due to their characteristic of being anti-GluR3 antibodies) and then tested in appropriate functional activity assays. In one specific embodiment, said activity is detected by elicited current evoked from GluR3-expressing oocytes in the presence of such autoantibodies.

In one embodiment, the serum level of anti-GluR3 antibodies with agonistic activity is considered abnormal when the agonistic activity detected with purified anti-GluR3 antibodies from a test sample is significantly higher, for example, at least 10%, 20%, 30%, 40%, 50%, 60%, 70%, 80%, 90% or at least 100% higher than the agonistic activity detected with antibodies from a control sample (e.g. serum samples from healthy donors or patients not suffering from Rasmussen's encephalitis or epilepsy).

In another embodiment, the serum level of anti-GluR3 antibodies with agonistic activity is considered abnormal when any significant agonistic activity is detected with purified anti-GluR3 antibodies from a test sample whereas no significant agonistic activity is detected in control samples (e.g. serum samples from healthy donors or patients not suffering from Rasmussen's encephalitis or epilepsy).

The usefulness of the 1H-quinazoline-2,4-diones of formula (I) in the treatment of the above-mentioned disorders can be confirmed in a range of standard tests including those indicated below.

### 1. Diagnosis of Rasmussen's Encephalitis

Methods for diagnosing Rasmussen's encephalitis are known. Diagnosis of Rasmussen's encephalitis is based on age of onset and clinico-pathological findings. Rasmussen's encephalitis typically manifest in the age of 14 months to 14 years. Most patients with Rasmussen's encephalitis have seizures as initial symptoms and at a later stage also hemiparesis, cognitive impairment and dysphasia. Radiological/pathological findings are typically a progressive, usually unilateral, cerebral atrophy; and an encephalitis-like pathological picture.

### 2. Diagnosis of Epilepsy

As epilepsy is a common chronic neurological disorder characterized by recurrent unprovoked seizures, methods for diagnosing this disorder are well-known.

### 3. Detection and Characterization of Anti-GluR3 Autoantibodies in Rasmussen's Encephalitis Patients or Epilepsy Patients

### 3.1 Example of a Physical Assay: ELISA for Binding to GluR3B

Enzyme Linked Immunosorbent Assay (ELISA) for detecting anti-GluR3 autoantibodies have been described in the art (see Cohen-Kashi Maline et al., Neurochem Res. 2006, 31, 1181-1190 or Wiendl et al., Neurobiology 2001, 57, 1511-1514).

For example, Immulon 96-well plates (Dynatech, Germany) are coated passively overnight at 4° C. with 50 μl of 20 μg/mL of free peptide in phosphate buffered saline (PBS)+0.05% Tween 20. After blocking unreacted sites of the plates with PBS+10% bovine serum albumin (BSA), serum (dilutions 1:10 to 1:10000) or cerebrospinal fluid (directly to 1:100) is added (100 μL/well) and incubated for 2 hours at 37° C. After washing vigorously, horseradish peroxidase-conjugated rabbit anti-human immunoglobulin (Ig) G antibody (Sigma, Germany) is added and detection is performed using 1004 of substrate (o-phenyldiamine [OPD] reagent, Abbott Diagnostics, Germany). Optical density (OD) at 450 nM can be reported.

Of course, different assays may be used, depending on whether IgG or IgM is being detected.

The following GluR3 peptides can be used:

(SEQ ID NO: 1, GluR3A1, aa 274-293)
NNENPMVQQFIQRWVRLDER,
(SEQ ID NO: 2, GluR3A1, aa 283-302)
FIQRWVRLDEREFPEAKNAP,
(SEQ ID NO: 3, GluR3B1, aa 400-419)
NEYERFVPFSDQQISNDSAS,
(SEQ ID NO: 4, GluR3B2, aa 404-423)
RFVPFSDQQISNDSASSENR,
(SEQ ID NO: 8, a variant of SEQ ID NO: 3)
NEYERFVPFSDQQISND,
(SEQ ID NO: 9)
NEYERFVPFSDQQISNDSASSENRL,
(SEQ ID NO: 10)
NEYERFVPFSDQQISNDSASSENRTIVVTT,
(SEQ ID NO: 11)
RKAGYWNEYERFVPFSDQQISNDSASSENR.

The following specificity control can be used: Class II-associated invariant chain peptide (SEQ ID NO:5, LPKPPKPVSKMRMATPLLMQALPM also called CLIP peptide, aa 97-120).

The cut-off for anti-GluR3 positivity in a patient suffering from Rasmussen's Encephalitis or epilepsy can be defined for example as mean OD±3 SD of the controls (e.g. healthy donors).

### 3.2 Example of a Functional Assay for Agonistic Activity: Whole Cell Current Evoked in GluR3 Expressing Oocvtes (Functional Assay for Determining Agonist-Like Activity of Anti-GluR3 Autoantibodies)

Agonistic activity of anti-GluR3 autoantibodies can be measured by detecting whole cell current recorded from voltage-clamped GluR3 expressing oocytes as described in detail in Neurochem Res (2006) 31:1181-1190.

Oocytes are obtained from commercial suppliers or are surgically removed from adult female Xenopus laevis frogs and defolliculated. Stage V-VI oocytes are selected and injected with about 25 ng (in 50n1) of cRNA transcripts from cDNA of rat or human GluR3. Electrophysiological studies are performed 3-7 days post-injection of the cRNA into the oocytes using a two-electrode voltage clamp system. The responses (currents) of GluR3 expressing oocytes with test purified anti-GluR3 antibodies can be compared with corresponding test concentrations of a known AMPA receptor agonist (as a positive control). As a negative control, similar concentrations of a purified IgG antibody can be used and anti-GluR3 antibodies can be tested on oocytes that do not express GluR3 to ensure the responses are mediated by the expressed GluR3.

4. Purification of Anti-GluR3 Autoantibodies from Patients

Purification of anti-GluR3 autoantibodies from serum samples of patients can be performed as described in Neurochem Res (2006) 31:1181-1190. Shortly, anti-GluR3 antibodies can be purified by affinity chromatography on an affinity column prepared using Affi-Gel 15 support (Bio-Red laboratories). GluR3B peptides are coupled to washed beads resuspended in a binding buffer. The gel is transferred to the column for use.

### 5. Assessment of Antagonism of Agonistic Activity of Anti-GluR3 Antibodies

Experiments may be performed on Xenopus oocytes as previously described in paragraph 3. Briefly, two electrode voltage clamp recordings may be performed from Xenopus laevis oocytes expressing GluR3 receptors. GluR3 activation may be accomplished by anti-GluR3 autoantibodies with agonistic activity purified from patients, antagonism of said activation may be performed by adding a 1H-quinazoline-2,4-dione of formula (I).

### 6. Clinical Testing: Improvement Trials for Rasmussen's Encephalitis

Characteristics/Symptoms of Rasmussen's encephalitis are described above. The improvement of such characteristics/symptoms can be measured in clinical trials. Clinical testing of the 1H-quinazoline-2,4-diones of formula (I) may be conducted, for example, in one of the following study designs. For example, the skilled physician may look at a number of aspects of patient behaviours and abilities. He will realise that such studies are considered as guidelines and the certain aspects of the studies may be modified and redefined depending on the circumstance and environment, for example.

### 6.1 Trial A: Normal Patient Population

A patient population, with a normal control is dosed 1-3 times per day for a week or longer tested. The test is designed to allow for improvement, i.e. that there is a measurable parameter increase of the impaired function. Patients are tested at the beginning and at the end of the dosage period and the results are compared and analyzed.

### 6.2 Trial B: Deficit Population

A patient population with a deficit associated with Rasmussen's encephalitis is dosed 1-3 times per day for a week or longer and tested. The test is designed to allow for improvement, i.e. that there is a measurable parameter increase of the impaired function. The patients are tested at the beginning and at the end of the dosage period and the results are compared and analyzed. Exemplary parameters to test could include fewer or absence of seizures, a reduction of cognitive impairment or a reduction of dysphasia. Also measurable could be visualization of the reversal of some of the neuronal structural defects (by imaging), such as a reduction of brain areas showing atrophy or a pathological picture suggesting encephalitis.

### 6.3 Considerations for Designing a Trial

When designing a trial, the skilled person will appreciate the need to protect both against floor and ceiling effects. In other words, the study designing should allow cognition to be measurably raised or lowered.
Conditions that artificially impair a function, e.g. cognition, are one way to test enhancement of that function. Such conditions are, for example, sleep deprivation and pharmacological challenges.
Placebo control is required for all trials.
In assessing the data, evaluation of the likelihood of learning and practice effects from repeat assessments must be made. The likelihood of such effects contaminating the data to produce false positives should be taken in to account when designing the test, e.g. the tests should not be identical (e.g. commit the same list of words to memory) but designed to study the same mechanism. Other countermeasures may include single testing at the end of a trial only.

### 7. Clinical Testing: Improvement Trials for Epilepsy

A patient population with diagnosis of partial seizures with or without generalization according to the seizure classification of the International League against Epilepsy is dosed 1-3 times per day for a week or longer and tested. The test is designed to allow improvements in seizure frequency or severity. Thus, patients are required to have had at least 4 or more partial seizures per month in the 3 months preceding the clinical trial and for the period of the trial no changes in their existing antiepileptic drug dosing regimen are allowed. The patient makes records of date and time of each seizure and description of the seizure type. Efficacy variables are the percent reduction in average weekly seizure rate, percent of seizure-free days and patient global impression of improvement.

In yet another embodiment, the invention relates to the use of a 1H-quinazoline-2,4-dione of formula (I) for the treatment, prevention or delay of progression of Rasmussen's encephalitis or epilepsy in a subject in need of such treatment, wherein said subject being selected among those having abnormal serum level of anti-GluR3 autoantibodies.

In yet another embodiment, the invention relates to a 1H-quinazoline-2,4-dione of formula (I) for the treatment, prevention or delay of progression of Rasmussen's encephalitis or epilepsy in a subject in need of such treatment, wherein said subject being selected among those having abnormal serum level of anti-GluR3 autoantibodies.

In yet another embodiment, the invention relates to a pharmaceutical composition comprising a 1H-quinazoline-2,4-dione of formula (I) for the treatment, prevention or delay of progression of Rasmussen's encephalitis or epilepsy in a subject in need of such treatment, wherein said subject being selected among those having abnormal serum level of anti-GluR3 autoantibodies.

In yet another embodiment, the invention relates to the use of a 1H-quinazoline-2,4-dione of formula (I) for the manufacture of a medicament for the treatment, prevention or delay of progression of Rasmussen's encephalitis or epilepsy in a subject in need of such treatment, wherein said subject being selected among those having abnormal serum level of anti-GluR3 autoantibodies.

In yet another embodiment, the invention relates to a kit comprising a 1H-quinazoline-2,4-dione of formula (I) and appropriate means for purifying and/or detecting anti-GluR3 autoantibodies from biological samples, for example, serum sample, and optionally, instructions for use in selecting patients with an abnormal serum level of anti-GluR3 autoantibodies, for the treatment of Rasmussen's encephalitis or epilepsy.

In yet another embodiment, the invention relates to the use of a 1H-quinazoline-2,4-dione of formula (I) for the treatment, prevention or delay of progression of Rasmussen's encephalitis or epilepsy in a subject in need of such treatment, wherein said subject being selected among those having abnormal serum level of anti-GluR3 autoantibodies with agonistic activity.

In yet another embodiment, the invention relates to a 1H-quinazoline-2,4-dione of formula (I) for the treatment, prevention or delay of progression of Rasmussen's encephalitis or epilepsy in a subject in need of such treatment, wherein said subject being selected among those having abnormal serum level of anti-GluR3 autoantibodies with agonistic activity.

In yet another embodiment, the invention relates to a pharmaceutical composition comprising a 1H-quinazoline-2,4-dione of formula (I) for the treatment, prevention or delay of progression of Rasmussen's encephalitis or epilepsy in a subject in need of such treatment, wherein said subject being selected among those having abnormal serum level of anti-GluR3 autoantibodies with agonistic activity.

In yet another embodiment, the invention relates to the use of a 1H-quinazoline-2,4-dione of formula (I) for the manufacture of a medicament for the treatment, prevention or delay of progression of Rasmussen's encephalitis or epilepsy in a subject in need of such treatment, wherein said subject being selected among those having abnormal serum level of anti-GluR3 autoantibodies with agonistic activity.

In yet another embodiment, the invention relates to a kit comprising a 1H-quinazoline-2,4-dione of formula (I) and appropriate means for purifying and/or detecting anti-GluR3 autoantibodies from biological samples, for example, serum sample, and optionally, instructions for use in selecting patients with an abnormal serum level of anti-GluR3 autoantibodies with agonistic activity.

### EXAMPLES

Two different ELISA assays were developed to detect either IgG autoantibodies or IgM autoantibodies in serum samples obtained from healthy volunteers or patients suffering from epilepsy.

### 1) ELISA for Anti-GluR3b IgG Autoantibodies

96-well flat-bottom streptavidin-coated plates (Nunc, Roskilde, DK) were washed three times with washing buffer before 100 μl of biotinylated peptide was pipetted into each well. Control experiments were performed using each serum in wells containing no peptide (NSB). Samples were also incubated with 100 μg/ml of peptide to specifically inhibit the signal. After 1 h-incubation at room temperature with 200 rpm-stirring, plates were washed. Human samples were diluted 1:10 in assay buffer (prepared fresh daily by mixing TBS-0.05% Tween20 (v/v) Buffer (TBST) with 2% SeaBlock blocking buffer (v/v) (Pierce Biotechnology Inc., Rockford, Ill.), 0.5 M MgCl₂,) and added to pre-determined wells at 100 μl/well, in duplicate. Samples were left to incubate on the plate at RT for 1.5h under 200 rpm-stirring. After the plate was washed 5 times with TBST, 100 μl of (HRP) labeled protein A (Sigma Aldrich, St. Louis, Mo.) 1:5000-diluted in Tris Tween was added to each well followed by incubation at RT for 1 h under stirring (200 rpm). Plates were washed 5 times with 400 μl/well of TBST, and HRP substrate (TMB horseradish peroxidase was added to the plate (100 μl/well)). Optical density (OD) at 450 nM was measured. Specific signal was obtained by subtracting NSB of each patient sample from the signal obtained with GluR3b peptide in the same patient sample. The inhibition assay using non biotinylated peptide preincubated with the sample to abolish the signal obtained with biotin-peptide was used as final confirmation for the positivity of the human sample against the GluR3b peptide.

### 2) ELISA for Anti-GluR3b IgM Autoantibodies

96-well flat-bottom plates (Nunc, Roskilde, Dk) were washed 5 times with 400 μl/well TBST before 100 μl of peptide at 2 μg/ml was pipetted into each well. Plates were then blocked with 5% BSA. Control experiments were performed using wells uncoated with peptide for all sera tested (to measure NSB). Samples were also incubated with 100 μg/ml of peptide to inhibit specifically the signal. Human samples were diluted from 1/10 to 1/100 in assay buffer containing 1% BSA and added to pre-determined wells at 100 μl/well, in duplicate. Samples were left to incubate on the plate at RT for 1.5h under 200 rpm-stirring. After the plate was washed 5 times, 100 μl of monoclonal anti human-IgM-AP (Sigma Aldrich, St. Louis, Mo.) 1:5000 diluted in assay buffer was added to each well followed by incubation at RT for 1 h under stirring (200 rpm). Plates were washed, and AP substrate (Ultima PNPP) was added to the plate (100 μl/well). Optical density (OD) at 405 nM was measured. Specific signal was obtained by subtracting NSB of each patient sample from the signal obtained with GluR3b peptide in the same patient sample. The inhibition assay using peptide pre-incubated with the sample to abolish the signal obtained with the coated peptide may be used for final confirmation of the positivity of the human sample against the GluR3b peptide.

For IgG autoantibody assay, the following two biotin labeled peptides (No 1 and 2) were used to coat the streptavidin plate, and peptide No. 5 (SEQ ID NO:6) was used as free peptide for the inhibition test.

For the IgM autoantibody assay, peptide No. 5 (SEQ ID NO:6) was used to coat the normal plate, and the same peptide (No 5) was used as free peptide for the inhibition test as well.

1. NEYERFVPFSDQQISNDSASSENRL-biotin
2. Biotin-NEYERFVPFSDQQISNDSASSENR
3. NEYERFVPFSDQQISNDSASSENRTIVVTT-Biotin
4. Biotin-RKAGYWNEYERFVPFSDQQISNDSASSENR
(SEQ ID NO: 6)
5. NEYERFVPFSDQQISNDSASSENR

### Results for IgG Screening of Patient Serum Samples

H017 is healthy volunteer negative control. H015 is healthy volunteer positive control

| | 2 ug/mL | |
|---|---|---|
| | Peptide | |
| | coated | Pos/Neg |
| Blank | 0.000 | |
| Neg | −0.010 | − |
| (H017) | | |
| 1:10 | | |
| Pos 50 | 1.290 | + |
| ng/ml in | | |
| 1:10 hsp | | |
| Pos + Pep | 0.017 | − |
| H015 1:10 | 0.548 | + |
| H015 | 0.000 | − |
| 1:10 + Pep | | |
| P001 1:10 | −0.004 | − |
| P001 | 0.000 | − |
| 1:10 + Pep | | |
| P002 1:10 | 0.029 | − |
| P002 | 0.002 | − |
| 1:10 + Pep | | |
| P003 1:10 | 0.004 | − |
| P003 | 0.002 | − |
| 1:10 + Pep | | |
| P004 1:10 | −0.012 | − |
| P004 | −0.039 | − |
| 1:10 + Pep | | |
| P005 1:10 | 0.007 | − |
| P005 | 0.005 | − |
| 1:10 + Pep | | |
| P006 1:10 | 0.040 | + |
| P006 | 0.003 | − |
| 1:10 + Pep | | |
| P007 1:10 | 0.008 | − |
| P007 | 0.007 | − |
| 1:10 + Pep | | |
| P009 1:10 | 0.007 | − |
| P009 | 0.005 | − |
| 1:10 + Pep | | |
| P010 1:10 | −0.015 | − |
| P010 | −0.006 | − |
| 1:10 + Pep | | |
| NCO | 0.033 | |
| Blank | 0.010 | |
| Neg | 0.000 | − |
| (H017) | | |
| 1:10 | | |
| Pos 50 | 1.399 | + |
| ng/ml in | | |
| 1:10 hsp | | |
| Pos + Pep | 0.020 | − |
| H015 1:10 | 0.593 | + |
| H015 | −0.006 | − |
| 1:10 + Pep | | |
| P011 1:10 | 0.000 | − |
| P011 | −0.003 | − |
| 1:10 + Pep | | |
| P012 1:10 | 0.003 | − |
| P012 | 0.002 | − |
| 1:10 + Pep | | |
| P013 1:10 | 0.102 | + |
| P013 | 0.098 | + |
| 1:10 + Pep | | |
| P014 1:10 | 0.005 | − |
| P014 | 0.006 | − |
| 1:10 + Pep | | |
| P015 1:10 | 0.006 | − |
| P015 | 0.004 | − |
| 1:10 + Pep | | |
| P016 1:10 | −0.002 | − |
| P016 | 0.001 | − |
| 1:10 + Pep | | |
| P021 1:10 | 0.014 | − |
| P021 | 0.009 | − |
| 1:10 + Pep | | |
| P022 1:10 | 0.009 | − |
| P022 | −0.004 | − |
| 1:10 + Pep | | |
| P023 1:10 | 0.003 | − |
| P023 | −0.001 | − |
| 1:10 + Pep | | |
| NCO | 0.043 | |

### Results for IgM Screening of Patient Serum Samples

H017 is healthy volunteer negative control. H015 is healthy volunteer positive control

| 2 ug/mL | | | 2 ug/mL | | | 2 ug/mL | | |
|---|---|---|---|---|---|---|---|---|
| Peptide | 1% | Pos/ | Peptide | 1% | Pos/ | Peptide | 1% | Pos/ |
| coated | BSA | Neg | coated | BSA | Neg | coated | BSA | Neg |
| Blank | 0.000 | | Blank | 0.000 | | Blank | 0.013 | |
| Pos Rab | 0.058 | − | Pos Rab | 0.068 | − | Pos Rab | 0.072 | − |
| Ser 1:50 | | | Ser 1:50 | | | Ser 1:50 | | |
| Pos + Pep | 0.001 | − | Pos + Pep | 0.006 | − | Pos + Pep | 0.003 | − |
| H015 | 0.321 | + | H015 1:50 | 0.359 | + | H015 1:50 | 0.343 | + |
| 1:50 | | | | | | | | |
| H015 | 0.254 | + | H015 | 0.285 | + | H015 | 0.256 | + |
| 1:50 + Pep | | | 1:50 + Pep | | | 1:50 + Pep | | |
| H017 | −0.009 | − | H017 1:50 | −0.001 | − | H017 1:50 | −0.006 | − |
| 1:50 | | | | | | | | |
| H017 | −0.013 | − | H017 | −0.003 | − | H017 | −0.005 | − |
| 1:50 + Pep | | | 1:50 + Pep | | | 1:50 + Pep | | |
| P001 1:50 | −0.022 | − | P010 1:50 | −0.004 | − | P018 1:50 | 0.120 | + |
| P001 | −0.028 | − | P010 | −0.014 | − | P018 | −0.002 | − |
| 1:50 + Pep | | | 1:50 + Pep | | | 1:50 + Pep | | |
| P002 1:50 | −0.002 | − | P011 1:50 | −0.003 | − | P019 1:50 | 0.016 | − |
| P002 | −0.014 | − | P011 | −0.007 | − | P019 | 0.008 | − |
| 1:50 + Pep | | | 1:50 + Pep | | | 1:50 + Pep | | |
| P003 1:50 | −0.001 | − | P012 1:50 | −0.040 | − | P020 1:50 | −0.007 | − |
| P003 | −0.005 | − | P012 | −0.048 | − | P020 | −0.008 | − |
| 1:50 + Pep | | | 1:50 + Pep | | | 1:50 + Pep | | |
| P004 1:50 | −0.083 | − | P013 1:50 | −0.003 | − | P021 1:50 | −0.026 | − |
| P004 | −0.079 | − | P013 | −0.008 | − | P021 | −0.024 | − |
| 1:50 + Pep | | | 1:50 + Pep | | | 1:50 + Pep | | |
| P005 1:50 | −0.011 | − | P014 1:50 | 0.001 | − | P022 1:50 | −0.025 | − |
| P005 | −0.008 | − | P014 | −0.003 | − | P022 | 0.084 | − |
| 1:50 + Pep | | | 1:50 + Pep | | | 1:50 + Pep | | |
| P006 1:50 | 0.068 | − | P015 1:50 | −0.001 | − | P023 1:50 | −0.006 | − |
| P006 | 0.040 | − | P015 | −0.008 | − | P023 | −0.020 | − |
| 1:50 + Pep | | | 1:50 + Pep | | | 1:50 + Pep | | |
| P007 1:50 | 0.073 | − | P016 1:50 | −0.006 | − | Blank | 0.000 | − |
| P007 | 0.051 | − | P016 | −0.006 | − | Blank | 0.005 | − |
| 1:50 + Pep | | | 1:50 + Pep | | | | | |
| P009 1:50 | 0.001 | − | P017 1:50 | 0.011 | − | Blank | −0.002 | − |
| P009 | −0.003 | − | P017 | −0.002 | − | Blank | −0.014 | − |
| 1:50 + Pep | | | 1:50 + Pep | | | | | |
| Blank | 0.013 | − | Blank | −0.010 | − | Blank | 0.005 | − |
| NCO | 0.114 | | NCO | 0.122 | | NCO | 0.117 | |

For the IgG assay, 21 healthy volunteer samples were screened to set the negative cut off value (NCO). 18 epilepsy patient samples were also screened. The positive controls of rabbit anti-GluR2Ab spiked in serum and healthy sample H015 showed high binding signal. Both of these signals, along with the positive signal from P006, could be significantly blocked using excess amounts of free peptide.

For the IgM assay, 22 healthy volunteer samples were screened to set the negative cut off value (NCO). 22 epilepsy patient samples were also screened. The positive control of healthy sample H015 could only partially be blocked with free peptide. However, epilepsy sample P018 showed a positive binding signal that could be completely blocked with free peptide.

### Oral Bioavailability of the Compounds of the Invention

Oral bioavailability of the compounds of the invention may be demonstrated using any generally known test in which the compound is administered orally and a biological effect observed.

Oral bioavailability of the compounds of the invention in the treatment of epilepsy or Rasmussen's encephalitis, e.g. Rasmussen's encephalitis, may be further quantified by the Maximal Electroshock test, which demonstrates that the compounds are orally bioavailable, penetrate the blood brain barrier and bind to the target receptor.

The oral bioavailability was tested using the audiogenic mouse test (Audiogenic seizures, R. L. Collins; Chapter 14, pages: 347-372. In: Experimental Models of Epilepsy; By: Pupura, Penry, Tower, Woodbury, Walter, Raven Press, New York, 1972. Standard Book Number: 0-911216-26-X) and/or the MES test. Where the MES test was used (as described below), the result is given in Table 1.

Table 1: In-Vivo Activity of Parent Compounds and Prodrugs in the Murine Maximal Electro Shock Test

Compounds of the invention were tested in OF1 mice using the maximal electroshock test (MES Test) described in detail by Schmutz et al., Naunyn-Schmiedeberg's Arch Pharmacol 1990, 342, 61-66. Briefly, generalized tonic-clonic convulsions of the hind extremities were induced by passing electrical current through temporal electrodes (50 Hz, 18 mA, 0.2s). Mice treated by vehicle showed mean seizure durations of 12-14s. 30 mg/kg carbamazepine was used as a positive control; mice were classified as protected by a compound if the duration of the seizure lasted only 3 second or less. Five mice were used for each treatment condition and the percentage of protected mice was used as readout (i.e. a compound could give 0%, 20%, 40%, 60%, 80% or 100% protection). Compounds of the invention were given at a dose of 50 mg/kg, p.o., 1 hour prior to induction of convulsions (i.e. “pre-treatment time—1 h”).

ED50 values (ED: effective dose) were calculated using GraphPad Prism, v4.02.

15 s after shock administration, mouse blood was collected for determination of compounds' blood exposure.

The results are shown below in Table 1.

TABLE 1

| | | | MES-Test | |
|---|---|---|---|---|
| | | In vivo | (1 h, po) | |
| | | orally | ED50 | |
| Compound | Structure | active | [mg/kg] | IUPAC name |
| A-1 | 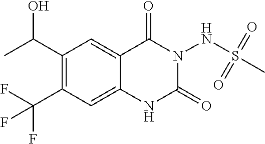 [SMILES: CC(O)c1cc2c(=O)n(NS(C)(=O)=O)c(=O)[nH]c2cc1C(F)(F)F] | Yes | 64 | N-[6-(1-Hydroxy-ethyl)-2,4-dioxo-7- trifluoromethyl-1,4-dihydro-2H-quinazolin- 3-yl]-methanesulfonamide |
| A-2 | 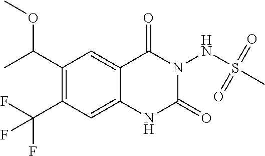 [SMILES: COC(C)c1cc2c(=O)n(NS(C)(=O)=O)c(=O)[nH]c2cc1C(F)(F)F] | Yes | 6.0 | N-[6-(1-Methoxy-ethyl)-2,4-dioxo-7- trifluoromethyl-1,4-dihydro-2H-quinazolin- 3-yl]-methanesulfonamide |
| A-3 | 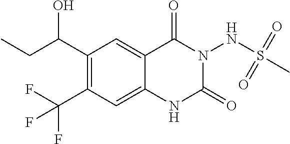 [SMILES: CCC(O)c1cc2c(=O)n(NS(C)(=O)=O)c(=O)[nH]c2cc1C(F)(F)F] | Yes | 19.6 | N-[6-(1-Hydroxy-propyl)-2,4-dioxo-7- trifluoromethyl-1,4-dihydro-2H-quinazolin- 3-yl]-methanesulfonamide |
| A-4 | 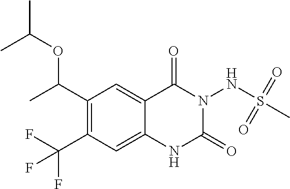 [SMILES: CC(C)OC(C)c1cc2c(=O)n(NS(C)(=O)=O)c(=O)[nH]c2cc1C(F)(F)F] | Yes | 15.6 | N-[6-(1-Isopropoxy-ethyl)-2,4-dioxo-7- trifluoromethyl-1,4-dihydro-2H-quinazolin- 3-yl]-methanesulfonamide |
| A-5 | 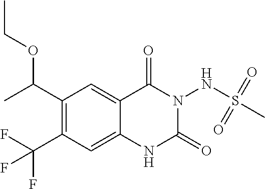 [SMILES: CCOC(C)c1cc2c(=O)n(NS(C)(=O)=O)c(=O)[nH]c2cc1C(F)(F)F] | Yes | 8.8 | N-[6-(1-Ethoxy-ethyl)-2,4-dioxo-7- trifluoromethyl-1,4-dihydro-2H-quinazolin- 3-yl]-methanesulfonamide |
| A-6 | 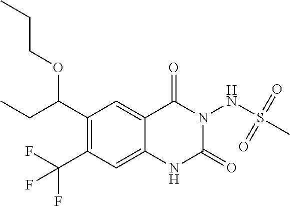 [SMILES: CCCOC(CC)c1cc2c(=O)n(NS(C)(=O)=O)c(=O)[nH]c2cc1C(F)(F)F] | Yes | nt¹ | N-[2,4-Dioxo-6-(1-propoxy-propyl)-7- trifluoromethyl-1,4-dihydro-2H-quinazolin- 3-yl]-methanesulfonamide |
| A-7 | 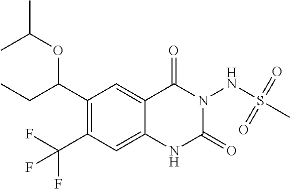 [SMILES: CCC(OC(C)C)c1cc2c(=O)n(NS(C)(=O)=O)c(=O)[nH]c2cc1C(F)(F)F] | Yes | 24.7 | N-[6-(1-isopropoxy-propyl)-2,4-dioxo-7- trifluoromethyl-1,4-dihydro-2H-quinazolin- 3-yl]-methanesulfonamide |
| A-8 | 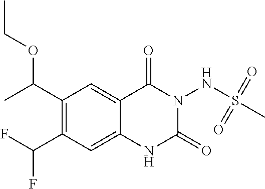 [SMILES: CCOC(C)c1cc2c(=O)n(NS(C)(=O)=O)c(=O)[nH]c2cc1C(F)F] | nt | nt | N-[7-Difluoromethyl-6-(1-ethoxy-ethyl)- 2,4-dioxo-1,4-dihydro-2H-quinazolin- 3-yl]-methanesulfonamide |
| A-9 | 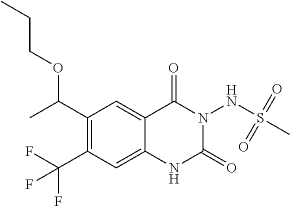 [SMILES: CCCOC(C)c1cc2c(=O)n(NS(C)(=O)=O)c(=O)[nH]c2cc1C(F)(F)F] | nt | nt | N-[2,4-Dioxo-6-(1-propoxy-ethyl)-7- trifluoromethyl-1,4-dihydro-2H-quinazolin- 3-yl]-methanesulfonamide |
| A-10 | 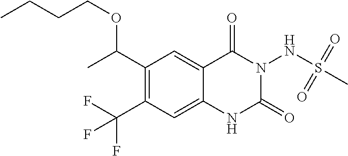 [SMILES: CCCCOC(C)c1cc2c(=O)n(NS(C)(=O)=O)c(=O)[nH]c2cc1C(F)(F)F] | nt | nt | N-[6-(1-Butoxy-ethyl)-2,4-dioxo-7- trifluoromethyl-1,4-dihydro-2H-quinazolin- 3-yl]-methanesulfonamide |
| A-11 | 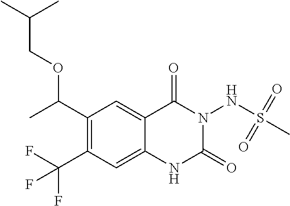 [SMILES: CC(C)COC(C)c1cc2c(=O)n(NS(C)(=O)=O)c(=O)[nH]c2cc1C(F)(F)F] | nt | nt | N-[6-(1-Isobutoxy-ethyl)-2,4-dioxo-7- trifluoromethyl-1,4-dihydro-2H-quinazolin- 3-yl]-methanesulfonamide |
| A-12 | 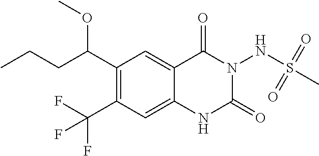 [SMILES: CCCC(OC)c1cc2c(=O)n(NS(C)(=O)=O)c(=O)[nH]c2cc1C(F)(F)F] | nt | nt | N-[6-(1-methoxy-butyl)-2,4-dioxo-7- trifluoromethyl-1,4-dihydro-2H-quinazolin- 3-yl]-methanesulfonamide |
| A-13 | 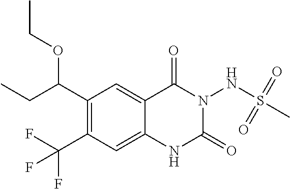 [SMILES: CCOC(CC)c1cc2c(=O)n(NS(C)(=O)=O)c(=O)[nH]c2cc1C(F)(F)F] | Yes | nt | N-[6-(1-Ethoxy-propyl)-2,4-dioxo-7- trifluoromethyl-1,4-dihydro-2H-quinazolin- 3-yl]-methanesulfonamide |
| A-14 | 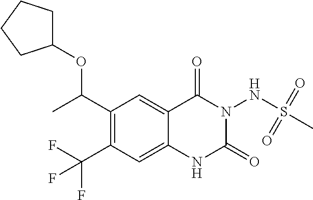 [SMILES: CC(OC1CCCC1)c1cc2c(=O)n(NS(C)(=O)=O)c(=O)[nH]c2cc1C(F)(F)F] | yes | nt | N-[6-(1-Cyclopentyloxy-ethyl)-2,4-dioxo-7- trifluoromethyl-1,4-dihydro-2H-quinazolin- 3-yl]-methanesulfonamide |
| A-15 | 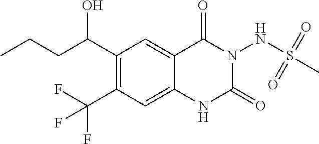 [SMILES: CCCC(O)c1cc2c(=O)n(NS(C)(=O)=O)c(=O)[nH]c2cc1C(F)(F)F] | Yes | 35 | N-[6-(1-Hydroxy-butyl)-2,4-dioxo-7- trifluoromethyl-1,4-dihydro-2H-quinazolin- 3-yl]-methanesulfonamide |
| A-16 | 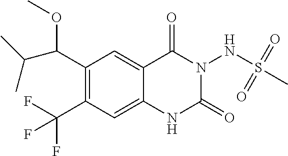 [SMILES: COC(c1cc2c(=O)n(NS(C)(=O)=O)c(=O)[nH]c2cc1C(F)(F)F)C(C)C] | nt | nt | N-[6-(1-Methoxy-2-methyl-propyl)-2,4-dioxo-7- trifluoromethyl-1,4-dihydro-2H-quinazolin- 3-yl]-methanesulfonamide |
| A-17 | 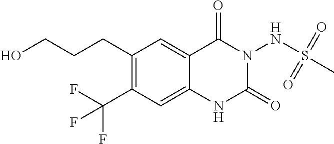 [SMILES: CS(=O)(=O)Nn1c(=O)[nH]c2cc(C(F)(F)F)c(CCCO)cc2c1=O] | nt | nt | N-[6-(3-Hydroxy-propyl)-2,4-dioxo-7- trifluoromethyl-1,4-dihydro-2H-quinazolin- 3-yl]-methanesulfonamide |
| A-18 | 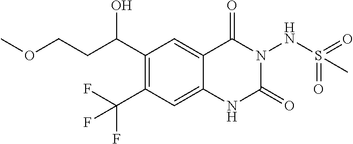 [SMILES: COCCC(O)c1cc2c(=O)n(NS(C)(=O)=O)c(=O)[nH]c2cc1C(F)(F)F] | Yes | nt | N-[6-(1-Hydroxy-3-methoxy-propyl)-2,4-dioxo-7- trifluoromethyl-1,4-dihydro-2H-quinazolin- 3-yl]-methanesulfonamide |
| A-19 | 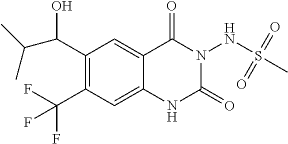 [SMILES: CC(C)C(O)c1cc2c(=O)n(NS(C)(=O)=O)c(=O)[nH]c2cc1C(F)(F)F] | nt | nt | N-[6-(1-Hydroxy-2-methyl-propyl)-2,4-dioxo-7- trifluoromethyl-1,4-dihydro-2H-quinazolin- 3-yl]-methanesulfonamide |
| B-1 | 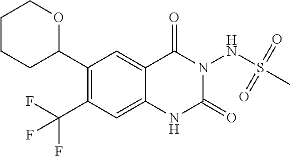 [SMILES: CS(=O)(=O)Nn1c(=O)[nH]c2cc(C(F)(F)F)c(C3CCCCO3)cc2c1=O] | nt | nt | N-[2,4-Dioxo-6-(tetrahydro-pyran-2-yl)-7- trifluoromethyl-1,4-dihydro-2H-quinazolin- 3-yl]-methanesulfonamide |
| B-2 | 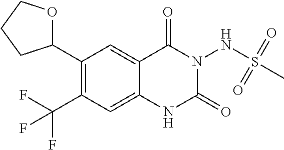 [SMILES: CS(=O)(=O)Nn1c(=O)[nH]c2cc(C(F)(F)F)c(C3CCCO3)cc2c1=O] | Yes | 12.8 (R)² 33.2 (S) | N-[2,4-Dioxo-6-(tetrahydro-furan-2-yl)-7- trifluoromethyl-1,4-dihydro-2H-quinazolin- 3-yl]-methanesulfonamide |
| B-3 | 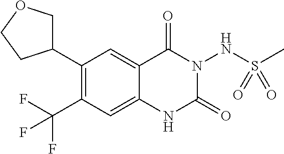 [SMILES: CS(=O)(=O)Nn1c(=O)[nH]c2cc(C(F)(F)F)c(C3CCOC3)cc2c1=O] | Yes | 20%@25³ | N-[2,4-Dioxo-6-(tetrahydro-furan-3-yl)-7- trifluoromethyl-1,4-dihydro-2H- quinazolin-3-yl]-methanesulfonamide |
| C-1 | 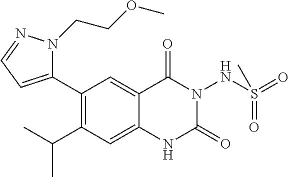 [SMILES: COCCn1nccc1-c1cc2c(=O)n(NS(C)(=O)=O)c(=O)[nH]c2cc1C(C)C] | yes | 40%@25 | N-{7-Isopropyl-6-[2-(2-methoxy-ethyl)-2H- pyrazol-3-yl]-2,4-dioxo-1,4-dihydro-2H- quinazolin-3-yl}-methanesulfonamide |
| C-2 | 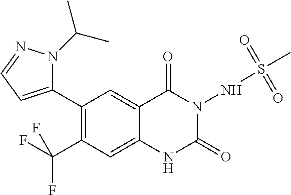 [SMILES: CC(C)n1nccc1-c1cc2c(=O)n(NS(C)(=O)=O)c(=O)[nH]c2cc1C(F)(F)F] | Yes | 17.7 | N-[6-(2-Isopropyl-2H-pyrazol-3-yl)-2,4-dioxo-7- trifluoromethyl-1,4-dihydro-2H-quinazolin- 3-yl]-methanesulfonamide |
| C-3 | 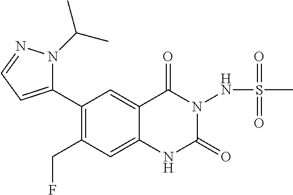 [SMILES: CC(C)n1nccc1-c1cc2c(=O)n(NS(C)(=O)=O)c(=O)[nH]c2cc1CF] | Yes | 13.5 | N-[7-Fluoromethyl-6-(2-isopropyl-2H-pyrazol-3- yl)-2,4-dioxo-1,4-dihydro-2H-quinazolin- 3-yl]-methanesulfonamide |
| C-4 | 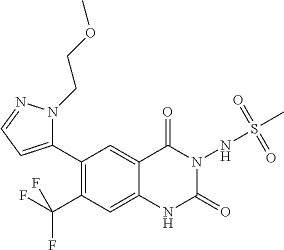 [SMILES: COCCn1nccc1-c1cc2c(=O)n(NS(C)(=O)=O)c(=O)[nH]c2cc1C(F)(F)F] | Yes | nt | N-{6-[2-(2-Methoxy-ethyl)-2H-pyrazol-3-yl]-2,4- dioxo-7-trifluoromethyl-1,4-dihydro-2H- quinazolin-3-yl}-methanesulfonamide |
| C-5 | 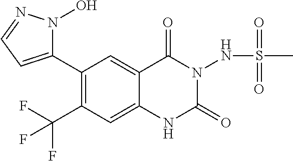 [SMILES: CS(=O)(=O)Nn1c(=O)[nH]c2cc(C(F)(F)F)c(-c3ccnn3O)cc2c1=O] | Yes | nt | N-[6-(2-Hydroxy-2H-pyrazol-3-yl)-2,4-dioxo-7- trifluoromethyl-1,4-dihydro-2H-quinazolin- 3-yl]-methanesulfonamide |
| C-6 | 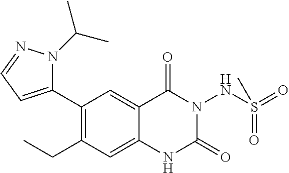 [SMILES: CCc1cc2[nH]c(=O)n(NS(C)(=O)=O)c(=O)c2cc1-c1ccnn1C(C)C] | Yes | 20%@50 | N-[7-Ethyl-6-(2-isopropyl-2H-pyrazol-3-yl)-2,4- dioxo-1,4-dihydro-2H-quinazolin-3-yl]- methanesulfonamide |
| C-7 | 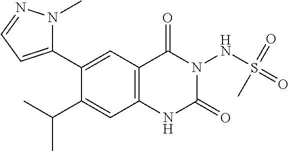 [SMILES: CC(C)c1cc2[nH]c(=O)n(NS(C)(=O)=O)c(=O)c2cc1-c1ccnn1C] | Yes | 6.9 | N-[7-Isopropyl-6-(2-methyl-2H-pyrazol-3-yl)-2,4- dioxo-1,4-dihydro-2H-quinazolin-3-yl]- methanesulfonamide |
| C-8 | 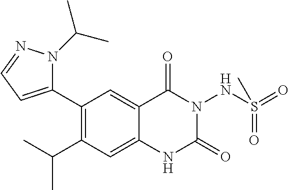 [SMILES: CC(C)c1cc2[nH]c(=O)n(NS(C)(=O)=O)c(=O)c2cc1-c1ccnn1C(C)C] | yes | 40%@50 | N-[7-Isopropyl-6-(2-isopropyl-2H-pyrazol-3-yl)- 2,4-dioxo-1,4-dihydro-2H-quinazolin-3-yl]- methanesulfonamide |
| C-9 | 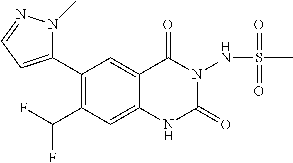 [SMILES: Cn1nccc1-c1cc2c(=O)n(NS(C)(=O)=O)c(=O)[nH]c2cc1C(F)F] | Yes | 7.5 | N-[7-Difluoromethyl-6-(2-methyl-2H-pyrazol-3-yl)- 2,4-dioxo-1,4-dihydro-2H-quinazolin-3-yl]- methanesulfonamide |
| C-10 | 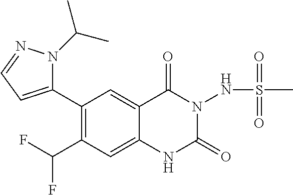 [SMILES: CC(C)n1nccc1-c1cc2c(=O)n(NS(C)(=O)=O)c(=O)[nH]c2cc1C(F)F] | Yes | 20.3 | N-[7-Difluoromethyl-6-(2-isopropyl-2H-pyrazol-3- yl)-2,4-dioxo-1,4-dihydro-2H-quinazolin-3-yl]- methanesulfonamide |
| C-11 | 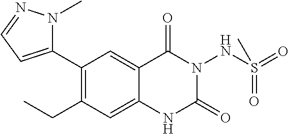 [SMILES: CCc1cc2[nH]c(=O)n(NS(C)(=O)=O)c(=O)c2cc1-c1ccnn1C] | Yes | 6.1 | N-[7-Ethyl-6-(2-methyl-2H-pyrazol-3-yl)-2,4- dioxo-1,4-dihydro-2H-quinazolin-3-yl]- methanesulfonamide |
| C-12 | 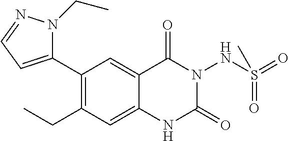 [SMILES: CCc1cc2[nH]c(=O)n(NS(C)(=O)=O)c(=O)c2cc1-c1ccnn1CC] | Yes | 42.8 | N-[7-Ethyl-6-(2-ethyl-2H-pyrazol-3-yl)-2,4-dioxo- 1,4-dihydro-2H-quinazolin-3-yl]- methanesulfonamide |
| C-13 | 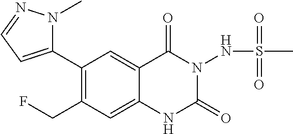 [SMILES: Cn1nccc1-c1cc2c(=O)n(NS(C)(=O)=O)c(=O)[nH]c2cc1CF] | nt | nt | N-[7-Fluoromethyl-6-(2-methyl-2H-pyrazol-3-yl)- 2,4-dioxo-1,4-dihydro-2H-quinazolin-3-yl]- methanesulfonamide |
| C-14 | 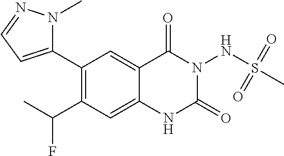 [SMILES: CC(F)c1cc2[nH]c(=O)n(NS(C)(=O)=O)c(=O)c2cc1-c1ccnn1C] | nt | nt | N-[7-(1-fluoro-ethyl)-6-(2-methyl-2H-pyrazol-3-yl)- 2,4-dioxo-1,4-dihydro-2H-quinazolin-3-yl]- methanesulfonamide |
| C-15 | 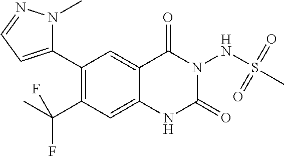 [SMILES: Cn1nccc1-c1cc2c(=O)n(NS(C)(=O)=O)c(=O)[nH]c2cc1C(C)(F)F] | Yes | 80%@20 | N-[7-(1,1-difluoro-ethyl)-6-(2-methyl-2H-pyrazol- 3-yl)-2,4-dioxo-1,4-dihydro-2H-quinazolin-3-yl]- methanesulfonamide |
| C-16 | 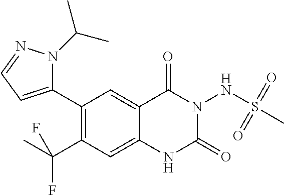 [SMILES: CC(C)n1nccc1-c1cc2c(=O)n(NS(C)(=O)=O)c(=O)[nH]c2cc1C(C)(F)F] | Yes | nt | N-[7-(1,1-difluoro-ethyl)-6-(2-isopropyl-2H- pyrazol-3-yl)-2,4-dioxo-1,4-dihydro-2H- quinazolin-3-yl]-methanesulfonamide |
| C-17 | 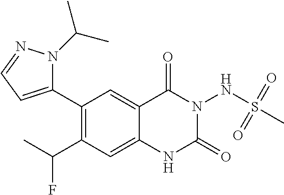 [SMILES: CC(F)c1cc2[nH]c(=O)n(NS(C)(=O)=O)c(=O)c2cc1-c1ccnn1C(C)C] | Yes | >20 | N-[7-(1-fluoro-ethyl)-6-(2-isopropyl-2H-pyrazol-3- yl)-2,4-dioxo-1,4-dihydro-2H-quinazolin-3-yl]- methanesulfonamide |
| C-18 | 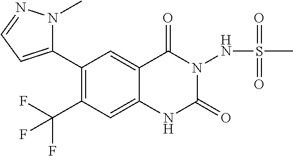 [SMILES: Cn1nccc1-c1cc2c(=O)n(NS(C)(=O)=O)c(=O)[nH]c2cc1C(F)(F)F] | Yes | 14.8 | N-[6-(2-Methyl-2H-pyrazol-3-yl)-2,4-dioxo-7- trifluoromethyl-1,4-dihydro-2H-quinazolin-3-yl]- methanesulfonamide |
| Comparative | 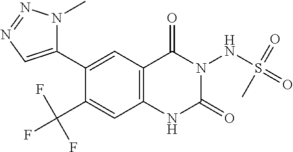 [SMILES: Cn1nncc1-c1cc2c(=O)n(NS(C)(=O)=O)c(=O)[nH]c2cc1C(F)(F)F] | No | 0%@50 | N-(6-(1-methyl-1H-1,2,3-triazol-5-yl)-2,4-dioxo-7- trifluoromethyl-1,4-dihydro-2H-quinazolin-3-yl]- methanesulfonamide |

¹The term “nt” throughout the table means “not tested”
²(R) and (S) indicate the two enantiomers.
³The term “20%@25” means 20% protection at 50 mg/kg.

This data shows that the compounds for use in the invention exhibit beneficial oral bioavailability relating to the comparative example (not in accordance with the invention).

1. A compound of formula (I);
wherein 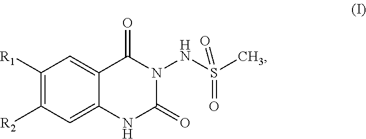
R₁ is C₁-C₆alkyl substituted by one, two or three substituents selected from hydroxy, C₁-C₆alkoxy or C₅-C₆cycloalkoxy; C₅-C₆cycloalkyl substituted by one, two or three substituents selected from hydroxy, C₁-C₆alkoxy or C₅-C₆cycloalkoxy; or
R₁ is 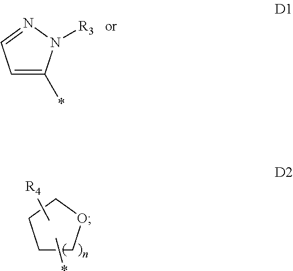
R₃ is C₁-C₆alkyl, hydroxy or C₁-C₆alkoxy-C₁-C₆alkyl;
R₄ is hydrogen or C₁-C₆alkyl;
n is 1 or 2;
R₂ is C₁-C₃alkyl or C₁-C₃-fluoroalkyl;
or a pharmaceutically acceptable salt or prodrug thereof;
for use in the treatment, prevention or delay of progression of Rasmussen's encephalitis.

2. A compound of formula (I) according to claim 1, wherein R₁ is D1 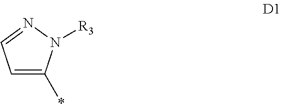
R₃ is C₁-C₆alkyl, hydroxy or C₁-C₆alkoxy-C₁-C₆alkyl;
R₂ is C₁-C₃alkyl or C₁-C₃-fluoroalkyl;
or a pharmaceutically acceptable salt or prodrug thereof;
for use in the treatment, prevention or delay of progression of Rasmussen's encephalitis.

3. A compound of formula (I) according to claim 1, wherein R₁ is D2 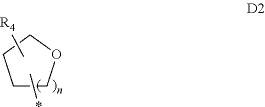
R₄ is hydrogen or C₁-C₆alkyl;
n is 1 or 2;
R₂ is C₁-C₃alkyl or C₁-C₃-fluoroalkyl;
or a pharmaceutically acceptable salt or prodrug thereof;
for use in the treatment, prevention or delay of progression of Rasmussen's encephalitis.

4. A compound of formula (I) for use according to claim 1, selected from the group consisting of:
N-[6-(1-Hydroxy-ethyl)-2,4-dioxo-7-trifluoromethyl-1,4-dihydro-2H-quinazolin-3-yl]-methanesulfonamide;
N-[6-(1-Methoxy-ethyl)-2,4-dioxo-7-trifluoromethyl-1,4-dihydro-2H-quinazolin-3-yl]-methanesulfonamide;
N-[6-(1-Hydroxy-propyl)-2,4-dioxo-7-trifluoromethyl-1,4-dihydro-2H-quinazolin-3-yl]-methanesulfonamide;
N-[6-(1-Isopropoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[2,4-Dioxo-6-(1-propoxy-propyl)-7-trifluoromethyl-1,4-dihydro-2H-quinazolin-3-yl]-methanesulfonamide;
N-[6-(1-isopropoxy-propyl)-2,4-dioxo-7-trifluoromethyl-1,4-dihydro-2H-quinazolin-3-yl]-methanesulfonamide;
N-[7-Difluoromethyl-6-(1-ethoxy-ethyl)-2,4-dioxo-1,4-dihydro-2H-quinazolin-3-yl]-methanesulfonamide;
N-[2,4-Dioxo-6-(1-propoxy-ethyl)-7-trifluoromethyl-1,4-dihydro-2H-quinazolin-3-yl]-methanesulfonamide;
N-[6-(1-Butoxy-ethyl)-2,4-dioxo-7-trifluoromethyl-1,4-dihydro-2H-quinazolin-3-yl]-methanesulfonamide;
N-[6-(1-Isobutoxy-ethyl)-2,4-dioxo-7-trifluoromethyl-1,4-dihydro-2H-quinazolin-3-yl]-methanesulfonamide;
N-[6-(1-methoxy-butyl)-2,4-dioxo-7-trifluoromethyl-1,4-dihydro-2H-quinazolin-3-yl]-methanesulfonamide;
N-[6-(1-Ethoxy-propyl)-2,4-dioxo-7-trifluoromethyl-1,4-dihydro-2H-quinazolin-3-yl]-methanesulfonamide;
N-[6-(1-Cyclopentyloxy-ethyl)-2,4-dioxo-7-trifluoromethyl-1,4-dihydro-2H-quinazolin-3-yl]-methanesulfonamide;
N-[6-(1-Hydroxy-butyl)-2,4-dioxo-7-trifluoromethyl-1,4-dihydro-2H-quinazolin-3-yl]-methanesulfonamide;
N-[6-(1-Methoxy-2-methyl-propyl)-2,4-dioxo-7-trifluoromethyl-1,4-dihydro-2H-quinazolin-3-yl]-methanesulfonamide;
N-[6-(3-Hydroxy-propyl)-2,4-dioxo-7-trifluoromethyl-1,4-dihydro-2H-quinazolin-3-yl]-methanesulfonamide;
N-[6-(1-Hydroxy-3-methoxy-propyl)-2,4-dioxo-7-trifluoromethyl-1,4-dihydro-2H-quinazolin-3-yl]-methanesulfonamide;
N-[6-(1-Hydroxy-2-methyl-propyl)-2,4-dioxo-7-trifluoromethyl-1,4-dihydro-2H-quinazolin-3-yl]-methanesulfonamide;
N-[2,4-Dioxo-6-(tetrahydro-pyran-2-yl)-7-trifluoromethyl-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[2,4-Dioxo-6-(tetrahydro-furan-3-yl)-7-trifluoromethyl-1,4-dihydro-2H-quinazolin-3-yl]-methanesulfonamide;
N-{7-Isopropyl-6-[2-(2-methoxy-ethyl)-2H-pyrazol-3-yl]-2,4-dioxo-1,4-dihydro-2H-quinazolin-3-yl}-methanesulfonamide;
N-[6-(2-Isopropyl-2,4pyrazol-3-yl)-2,4-dioxo-7-trifluoromethyl-1,4-dihydro-2H-quinazolin-3-yl]-methanesulfonamide;
N-[7-Fluoromethyl-6-(2-isopropyl-2H-pyrazol-3-yl)-2,4-dioxo-1,4-dihydro-2H-quinazolin-3-yl]-methanesulfonamide;
N-{6-[2-(2-Methoxy-ethyl)-2H-pyrazol-3-yl]-2,4-dioxo-7-trifluoromethyl-1,4-dihydro-2H-quinazolin-3-yl}-methanesulfonamide;
N-[6-(2-Hydroxy-2H-pyrazol-3-yl)-2,4-dioxo-7-trifluoromethyl-1,4-dihydro-2H-quinazolin-3-yl]-methanesulfonamide;
N-[7-Ethyl-6-(2-isopropyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-isopropyl-2H-pyrazol-3-yl)-2,4-dioxo-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
N-[7-Ethyl-6-(2-ethyl-2H-pyrazol-3-yl)-2,4-dioxo-1,4-dihydro-2H-quinazolin-3-yl]-methanesulfonamide;
N-[7-Fluoromethyl-6-(2-methyl-2H-pyrazol-3-yl)-2,4-dioxo-1,4-dihydro-2H-quinazolin-3-yl]-methanesulfonamide;
N-[7-(1-fluoro-ethyl)-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-isopropyl-2H-pyrazol-3-yl)-2,4-dioxo-1,4-dihydro-2H-quinazolin-3-yl]-methanesulfonamide;
N-[7-(1-fluoro-ethyl)-6-(2-isopropyl-2H-pyrazol-3-yl)-2,4-dioxo-1,4-dihydro-2H-quinazolin-3-yl]-methanesulfonamide; or
N-[6-(2-Methyl-2H-pyrazol-3-yl)-2,4-dioxo-7-trifluoromethyl-1,4-dihydro-2H-quinazolin-3-yl]-methanesulfonamide;
or a pharmaceutically acceptable salt or prodrug thereof.

5. A compound of formula (I) for use according to claim 1, selected from the group consisting of:
N-[6-(1-Methoxy-ethyl)-2,4-dioxo-7-trifluoromethyl-1,4-dihydro-2H-quinazolin-3-yl]-methanesulfonamide;
N-[6-(1-Hydroxy-propyl)-2,4-dioxo-7-trifluoromethyl-1,4-dihydro-2H-quinazolin-3-yl]-methanesulfonamide;
N-[6-(1-Isopropoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[6-(2-Isopropyl-2H-pyrazol-3-yl)-2,4-dioxo-7-trifluoromethyl-1,4-dihydro-2H-quinazolin-3-yl]-methanesulfonamide;
N-[7-Fluoromethyl-6-(2-isopropyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-methyl-2H-pyrazol-3-yl)-2,4-dioxo-1,4-dihydro-2H-quinazolin-3-yl]-methanesulfonamide;
N-[6-(2-Methyl-2H-pyrazol-3-yl)-2,4-dioxo-7-trifluoromethyl-1,4-dihydro-2H-quinazolin-3-yl]-methanesulfonamide;
or a pharmaceutically acceptable salt or prodrug thereof.

6. A compound of formula (I) for use according to claim 1, selected from the group consisting of:
N-[6-(1-Methoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
or a pharmaceutically acceptable salt or prodrug thereof.

7. A compound of formula (I) for use according to claim 1, wherein the compound of formula (I) is N-[7-Isopropyl-6-(2-methyl-2H-pyrazol-3-yl)-2,4-dioxo-1,4-dihydro-2H-quinazolin-3-yl]-methanesulfonamide, or a pharmaceutically acceptable salt or prodrug thereof.

8. A compound of formula (I) for use according to claim 1, wherein the compound of formula (I) is N-[7-Isopropyl-6-(2-methyl-2H-pyrazol-3-yl)-2,4-dioxo-1,4-dihydro-2H-quinazolin-3-yl]-methanesulfonamide.

9. A method for the treatment, prevention or delay of progression of Rasmussen's encephalitis in a subject in need of such treatment, which comprises administering to said subject a therapeutically effective amount of a compound of formula (I) as defined in claim 1 or a pharmaceutically acceptable salt or prodrug thereof.

10. A pharmaceutical composition comprising a compound of formula (I) as defined in claim 1 or a pharmaceutically acceptable salt or prodrug thereof, for the treatment, prevention or delay of progression of Rasmussen's encephalitis.

11-23. (canceled)
